(19) United States
(12) Patent Application Publication
(10) Pub. No.: US 2006/0064092 A1
(43) Pub. Date: Mar. 23, 2006

(54) Selective axis serrated rod low profile spinal fixation system

(76) Inventor: Robert S. Howland, Seal Beach, CA (US)

Correspondence Address: ORRICK, HERRINGTON & SUTCLIFFE, LLP;IP PROSECUTION DEPARTMENT, 4 PARK PLAZA, SUITE 1600, IRVINE, CA, 92614-2558, US

(21) Appl. No.: 11/216,876

(22) Filed: Aug. 30, 2005

Related U.S. Application Data
(63) Continuation-in-part of application No. 10/698,087, filed on Oct. 31, 2003.
Application No. 10/698,087 is a continuation-in-part of application No. 10/133,310, filed on Apr. 24, 2002, now Pat. No. 6,770,075.
(63) Continuation-in-part of application No. 11/133,708, filed on May 19, 2005.
Application No. 11/133,708 is a continuation-in-part of application No. 10/889,754, filed on Jul. 12, 2004.
Application No. 10/889,754 is a continuation-in-part of application No. 10/698,087, filed on Oct. 31, 2003.
Application No. 10/698,087 is a continuation-in-part of application No. 10/133,310, filed on Apr. 24, 2002, now Pat. No. 6,770,075.
Application No. 10/133,310 is a continuation-in-part of application No. 09/861,278, filed on May 17, 2001, now Pat. No. 6,478,798.

(51) Int. Cl. A61F 2/30

(52) U.S. Cl. 606/61

(57) ABSTRACT

A spinal fixation system includes a plurality of anchor screw assemblies, e.g. including anchor screws and clamp assemblies defining rod passages therethrough. A rod is receivable in the rod passages between the anchor screw assemblies, and a spacer is securable on the rod. The rod is preferably serrated over less than about 25% of the peripheral surface thereof, and the clamp assemblies preferably include engagement surfaces that include similarly spaced and oriented serrations. During use, a first anchor screw is screwed into a first vertebra, and a second anchor screw is screwed into a second vertebra adjacent the first vertebra, and clamp assemblies are mounted to each anchor screw. A rod is secured between the anchor screw assemblies, thereby fixing a relative spacing of the first and second vertebrae. A spacer may be crimped onto the exposed portion of the rod between the anchor screw assemblies, the spacer extending between the anchor screw assemblies to prevent the anchor screw assemblies, and, consequently, the first and second vertebrae, from moving towards one another.

This application is a continuation-in-part of U.S. patent application Ser. No. 10/698,087, filed Oct. 31, 2003, which application is a continuation-in-part of U.S. patent application Ser. No. 10/133,310, filed Apr. 24, 2002. This application is also a continuation-in-part of U.S. patent application Ser. No. 11/133,708, filed May 19, 2005, which application is a continuation-in-part of U.S. patent application Ser. No. 10/889,754, filed Jul. 12, 2004, which application is a continuation-in-part of U.S. patent application Ser. No. 10/698,087, filed Oct. 31, 2003, which application is a continuation-in-part of U.S. patent application Ser. No. 10/133,310, filed Apr. 24, 2002, now U.S. Pat. No. 6,770,075, which application is a continuation-in-part of U.S. patent application Ser. No. 09/861,278, filed May 17, 2001, now U.S. Pat. No. 6,478,798. The disclosures of each of the foregoing patent applications are expressly incorporated herein by reference.

### FIELD OF THE INVENTION

The present invention relates generally to apparatus and methods for treating spinal disorders, and more particularly to spinal fixation systems that may be secured between adjacent anchor screw assemblies, and methods for stabilizing, adjusting, or otherwise fixing adjacent vertebrae using such spinal fixation systems.

### BACKGROUND OF THE INVENTION

Various systems and methods have been suggested for treating spinal disorders, such as degenerative discs, stenosis, trauma, scoliosis, kyphosis, or spondylolisthesis. For example, U.S. Pat. No. 5,545,166, discloses a spinal fixation system that includes a plurality of anchor screws, clamp assemblies, pivot blocks, clamp blocks, and rods that are implanted along a patient's spine to fix two or more adjacent vertebrae relative to one another. The system generally includes a swing bolt anchor screw, a pivot block receivable on the swing bolt, and a clamp block receiving a rod therethrough that is pivotally attachable to the pivot block. In addition, the system includes one or more fixed anchor screws, and clamp assemblies for receiving the rod therein. The clamp assemblies and pivot block are receivable on the anchor screws by spindles that thread along a threaded portion of the anchor screws.

During use, vertebrae to be treated are surgically exposed, and an arrangement of anchor screws and clamp accessories are selected. For example, a fixed anchor screw may be screwed into each of the vertebrae on either side of a first vertebra. A rod is selected that may extend between the fixed anchor screws and that may be bent to conform to the shape of the anatomy encountered. The rod is inserted through a loose clamp block, and the rod is placed in clamp assemblies that are received over the fixed anchor screws.

A swing bolt anchor screw is then screwed into the first vertebra adjacent the rod, and a pivot block is received on the swing bolt screw. The clamp block and/or pivot block are adjusted such that the clamp block may be engaged with a pivot on the pivot block. A set screw may then be screwed into the clamp block to secure the clamp block to the pivot. A pair of set screws are also screwed into the clamp block to secure the rod within the clamp block. Preferably, a pair of such systems are implanted on either side of the vertebrae.

During the procedure, it may be desirable to adjust the vertebrae relative to one another. Once the system(s) is(are) connected as described above, the set screws may be loosened and the rod(s), clamp block(s), and/or pivot block(s) may be adjusted, e.g., by moving the spindle(s) to adjust the height of the pivot block(s) and/or clamp assemblies on the anchor screws, by pivoting the swing bolt anchor screw(s), and/or pivoting the clamp block(s) relative to the pivot block(s). Once the vertebrae have been moved into a desired position, the set screws may be tightened, and the spindles secured in position by crimping the walls surrounding the spindles.

An advantage of this system is that the swing bolt anchor screw, pivot block, and clamp block arrangement allows the system to be adjusted about two axes, i.e., the axis of the swing bolt anchor screw and the axis of the pivot on the pivot block. However, because the system of the '166 patent is polyaxial, i.e., may pivot about multiple axes, there is greater risk of the system coming out of alignment when the patient resumes normal physical activity.

This system is also very complicated, involving six parts, including three set screws, that are mounted on each swing bolt anchor screw. In addition, because the swing bolt is threaded, an intricate spindle device is required in order to allow the pivot block and clamp assemblies to be threaded onto the swing bolt, and still control their orientation about the axis of the swing bolt. Thus, because of its complexity and many intricate parts, this system may be expensive to manufacture and/or difficult to implant.

Accordingly, apparatus and methods for stabilizing, adjusting, and/or fixing vertebrae would be considered useful.

### SUMMARY OF THE INVENTION

The present invention is directed to spinal fixation systems that may be secured between adjacent anchor screw assemblies, e.g., to rods extending between the anchor screw assemblies, and to methods for stabilizing, adjusting, or otherwise fixing adjacent vertebrae using such spinal fixation systems.

In accordance with one aspect of the present invention, a spinal fixation system is provided that includes a first anchor screw assembly including a first passage and a first screw, the first screw having a threaded portion configured to be screwed into a first vertebra, and a second anchor screw assembly including a second passage and a second screw, the second screw having a threaded portion configured to be screwed into a second vertebra adjacent the first vertebra. In an exemplary embodiment, one or both of the anchor screw assemblies may include a saddle or clamp assembly receivable on the respective screw, each saddle assembly including a rod passage therethrough defining the first and second passages. Preferably, the saddle assemblies include upper and lower saddles or clamp portions that may together define the rod passage.

A rod or other elongate member is receivable in the first and second passages, the elongate member including an exposed portion extending between the first and second anchor screw assemblies. A spacer is securable on the exposed portion of the elongate member, the spacer having a length substantially similar to a length of the exposed portion of the elongate member for preventing the first and second anchor screw assemblies from moving towards one another.

In several embodiments, the rod or other elongate member includes longitudinal serrations over at least a portion of the peripheral surface of the rod or elongate member. In several preferred embodiments, the serrations extend over less than about 25%, and preferably less than about 10% of the peripheral surface of the rod or elongate member. Similarly, the saddle or clamp assemblies preferably include mating serrations formed on the engagement surfaces of those members. In those embodiments in which the serrations extend over only a limited amount of the peripheral surface of the rod or elongate member, the engagement surfaces of the saddles or clamps may advantageously include serrations over only a similarly limited amount of the engagement surfaces.

In accordance with another aspect of the present invention, a kit is provided for stabilizing vertebrae relative to one another. Generally, the kit includes one or more substantially rigid rods (including rods having no serrations, rods having serrations over at least a portion of the peripheral surface of the rod, and/or fully serrated rods), and a plurality of “C” shaped spacers having a plurality of lengths, the spacers including opposing edges defining a pocket therebetween for receiving the one or more rods therein. The kit may also include a plurality of anchor screw assemblies, the anchor screw assemblies including anchor screws and a plurality of clamp assemblies for receiving the one or more rods therein.

Optionally, the kit may also include a tool for crimping at least a portion of the opposing edges of the spacers around the one or more rods to secure the spacers to the rods. In another option, the kit may include an apparatus for bending the one or more rods, e.g., to conform substantially to a natural curvature of a patient's spinal column being treated. In yet another option, the kit may include one or more flat, elongated, “plate” shaped connecting beams, such as those described in co-pending application Ser. No. 11/733,708.

In accordance with still another aspect of the present invention, a method is provided for stabilizing vertebrae relative to one another, the vertebrae being disposed adjacent one another along a central spinal axis. A first anchor screw may be screwed into a first vertebra, and a second anchor screw may be screwed into a second vertebra adjacent the first vertebra. A rod or other elongate member may be secured between the first and second anchor screws, e.g., using clamp assemblies, thereby fixing a relative distance of the first and second vertebrae. In several preferred embodiments, the rod or other elongate member includes longitudinal serrations extending over a limited portion of the peripheral surface thereof.

A spacer, e.g., a “C” shaped clip, may be secured or otherwise placed on the elongate member, e.g., by crimping the spacer around the elongate member. Preferably, the spacer extends substantially an entire length of the elongate member that is exposed between the first and the second anchor screws to prevent the first and second anchor from moving towards one another. For example, the spacer may abut clamp assemblies on the first and second anchor screws, thereby preventing the clamp assemblies, and consequently the anchor screws, from moving substantially towards one another. One or both of the clamp assemblies may have a tapered side portion to enhance abutment of the spacer and the clamp assemblies if the elongate member is bent, e.g., to conform to the natural curvature of the anatomy encountered.

In accordance with yet another aspect of the present invention, an anchor screw assembly is provided that includes a screw having a first threaded portion, and a second head portion. A swing bolt is pivotally coupled to the second portion of the screw. The swing bolt defines a first axis, and includes a noncircular region extending along the first axis, the noncircular region having a noncircular cross-section and a substantially smooth wall. In addition, the swing bolt may include a threaded region on its end opposite the screw.

A clamp assembly may be provided that includes first and second clamp portions that are receivable on the swing bolt. Each clamp portion has a noncircular first passage therethrough for receiving the noncircular region of the swing bolt therethrough. Thus, the noncircular region and the first passage have like cross-sections, thereby preventing rotation of the clamp assembly with respect to the swing bolt about the first axis when the noncircular region of the swing bolt is received in the first passages.

In addition, the first and second clamp portions have cooperating grooves therein, the cooperating grooves together defining a second passage extending along a second axis substantially transversely to the first axis when the first and second clamp portions are received on the swing bolt.

A fastener, e.g., a nut, is also provided for securing the clamp assembly on the swing bolt, e.g., that may be threaded onto the threaded region of the swing bolt to secure the clamp assembly on the swing bolt. In a preferred embodiment, the second portion of the screw includes a shoulder, and the clamp assembly may substantially engage the shoulder when the clamp assembly is fully secured on the swing bolt, thereby preventing the swing bolt from pivoting with respect to the screw.

In accordance with still another aspect of the present invention, a spinal fixation system is provided that includes a first anchor screw assembly, such as that described above. The first anchor screw assembly includes a first screw having a threaded portion, and a swing bolt pivotally coupled to the screw and including a noncircular region. The spinal fixation system also includes a plurality of clamp assemblies, including a first passage for receiving the first swing bolt therethrough, and a second passage for receiving an elongate member, e.g., a substantially rigid rod, therethrough. The dimensions of each clamp assembly may be different, e.g., including a second passage that is at one of a plurality of distances from the first passage and/or that is oriented at a predetermined angle along the clamp assembly. A fastener may be used for securing a selected clamp assembly on the swing bolt. Thus, when the selected clamp assembly is received on the first swing bolt, the first clamp assembly is fixed in a predetermined orientation with respect to a first pivot axis of the first swing bolt.

The spinal fixation system also includes a second anchor screw assembly including a second screw having a threaded portion and a hub, and a second selected clamp assembly receivable on the hub. The second screw may be a fixed screw or, preferably, a swing bolt anchor screw, similar to that described above. The second clamp assembly includes a third passage therethrough along a third axis. The second screw assembly may be oriented, when implanted, such that the third axis is substantially transverse to the first axis. Optionally, additional anchor screw assemblies may also be provided.

In accordance with another aspect of the present invention, a method is provided for simple alignment or otherwise stabilizing vertebrae relative to one another using a plurality of swing bolt anchor screw assemblies, such as those described above. A threaded portion of a first swing bolt anchor screw is screwed into a first vertebra until a first pivot axis of the first swing bolt anchor screw is generally parallel to the spinal axis. A threaded portion of a second swing bolt anchor screw is screwed into a second vertebra adjacent the first vertebra until a second pivot axis of the second swing bolt anchor screw is substantially transverse to the first pivot axis. If desired, a third anchor screw (or more) may be screwed into other vertebra adjacent to the first vertebra.

An angle of one or more swing bolts on the first and second swing bolt anchor screws may be adjusted about the first and second pivot axes. Lower clamp portions may be placed on the swing bolts of the first and second swing bolt anchor screws, either before or after the angle adjustments described above. A rod may be placed on the lower clamp portions, e.g., when the grooves in the lower clamp portions have been properly aligned with one another. Thus, the rod may extend between the first and second anchor screws, and between any additional anchor screws added generally in a straight line. In addition, if desired, the rod may be bent, e.g., in a single plane, to a predetermined configuration based upon anatomy encountered before securing the rod on the swing bolts.

Upper clamp portions may be secured on the swing bolts of the first and second swing bolt anchor screws, thereby securing the rod between the upper and lower clamp portions. For example, a nut or other fastener may be threaded onto the swing bolt after the upper and lower clamp portions, thereby securing the rod between the upper and lower clamp portions and/or securing the clamp assemblies on the swing bolts. These fasteners may also be loosened to allow adjustment of the vertebrae relative to one another, and then the fasteners may again be tightened to fix the vertebrae in desired relative positions. Optionally, a spacer, such as that described above, may be secured between the clamp assemblies to prevent movement of the swing bolts towards one another.

In accordance with another aspect of the present invention, a spinal fixation system is provided that includes a plurality of anchor assemblies, such as those described above. Each anchor screw assembly preferably includes a screw having a threaded portion, and a swing bolt pivotally coupled to the screw. The spinal fixation system also includes a plurality of clamp assemblies, each including a first passage for receiving a swing bolt therethrough, and a second passage for receiving an elongate member, e.g., a substantially rigid rod, therethrough.

The spinal fixation system also includes one or more cross-linking members extending between and interconnecting two or more elongate members, two or more anchor screw assemblies, or combinations thereof. The cross-linking members may include one or more brace members and one or more connecting members.

Other objects and features of the present invention will become apparent from consideration of the following description taken in conjunction with the accompanying drawings.

### BRIEF DESCRIPTION OF THE DRAWINGS

FIG. 1A is an exploded side view of a spinal fixation system, in accordance with the present invention.

FIG. 1B is a side view of the system of FIG. 1A implanted to stabilize a plurality of vertebrae.

FIG. 2A is a perspective view of a spacer clip, in accordance with the present invention.

FIG. 2B is a side view of a crimping tool for use with the spacer clip of FIG. 2A.

FIG. 3A is a perspective view of an embodiment of an anchor screw assembly, in accordance with the present invention.

FIG. 3B is an exploded perspective view of the anchor screw assembly of FIG. 3A.

FIGS. 4A and 4B are perspective and side views, respectively, of a screw for the anchor screw assembly of FIGS. 3A and 3B.

FIGS. 5A-5C are perspective and first and second side views, respectively, of a swing bolt for the anchor screw assembly of FIGS. 3A and 3B.

FIGS. 6A-6C are perspective and first and second side views, respectively, of an assembled screw and swing bolt for the anchor screw assembly of FIGS. 3A and 3B.

FIGS. 7A-7C are perspective and first and second side views, respectively, of a first embodiment of a lower clamp portion for a clamp assembly, in accordance with the present invention.

FIGS. 8A-8C are perspective and first and second side views, respectively, of a first embodiment of an upper clamp portion for a clamp assembly, in accordance with the present invention.

FIGS. 9A-9E are perspective views of alternative embodiments of a lower clamp portion, in accordance with the present invention.

FIGS. 10A-10E are perspective views of alternative embodiments of an upper clamp portion, in accordance with the present invention.

FIGS. 11A and 11B are perspective and side views, respectively, of another embodiment of an anchor screw, in accordance with the present invention.

FIGS. 12A-12C show a spinal fixation system being implanted between vertebrae of a patient, in accordance with the present invention.

FIG. 13 shows a pair of spinal fixation systems implanted along a patient's spine, in accordance with the present invention.

FIG. 14 is a perspective view of another preferred embodiment of an anchor screw assembly, in accordance with the present invention.

FIGS. 15A and 15B are top and front views, respectively, of an upper clamp portion for use with the anchor screw assembly of FIG. 14.

FIG. 15C is a front view of an alternative embodiment of an upper clamp portion for use with the anchor screw assembly of FIG. 14.

FIGS. 16 through 19 are similar views of a particularly preferred multi-axial or selective axial embodiment of the swing anchor screw assembly

FIGS. 20-21 illustrate alternative clamp assemblies for use with the swing anchor screw assembly of FIG. 16.

FIG. 22 is a perspective view of a spinal fixation system implanted to stabilize a plurality of vertebrae.

FIGS. 23A-B are perspective views of single rod spinal fixation systems, in accordance with the present invention.

FIGS. 24A-B are perspective views of double rod spinal fixation systems, in accordance with the present invention.

FIG. 25 is a perspective view of an anchor screw assembly.

FIG. 26 is a perspective view of a swing bolt.

FIG. 27 is a perspective view of a pin for attaching a swing bolt to an anchor screw.

FIGS. 28A-B are top and cross-sectional side views of a jam nut.

FIGS. 29A-F are perspective, side, and close-up views of clamp assemblies and portions thereof.

FIGS. 30A-B are perspective and side views of a clamp assembly.

FIGS. 31A-B are perspective and side views of another clamp assembly.

FIGS. 32A-B are perspective views of a pair of double-rod clamp assemblies.

FIGS. 33A-F are perspective, side, and close-up views of clamp assemblies and portions thereof.

FIGS. 34A-B are side and end views of a serrated rod.

FIGS. 35A-C are side, end, and cross-sectional views of a partially serrated rod.

FIGS. 36A-B are side and cross-sectional views of a partially serrated rod having a pre-formed bend.

FIGS. 37A-B are side and cross-sectional views of another partially serrated rod having a pre-formed bend.

FIG. 38 is a perspective view of a spinal fixation system including end cross-linking members.

FIG. 39 is a perspective view of another spinal fixation system including mid cross-linking members.

FIGS. 40A-C are perspective, side, and top views of a mid cross-linking member.

FIG. 41 is a perspective view of an upper clamp portion.

FIGS. 42A-B are perspective and close-up views of a pair of serrated cross links.

FIGS. 43A-C are perspective views of, respectively, a center clamp stud, a sliding clamp stud, and a clamp pin.

FIGS. 44A-B are perspective and side views of a lower clamp cross link. A clamp pin is also shown in FIG. 44B.

FIGS. 45A-B are perspective and side views of another lower clamp cross link. A clamp pin is also shown in FIG. 45B.

FIGS. 46A-B are perspective and side views of a lower clamp cross link having a 15° bend. A clamp pin is also shown in FIG. 46B.

FIGS. 47A-B are perspective and side views of another lower clamp cross link having a 15° bend. A clamp pin is also shown in FIG. 47B.

FIG. 48 is a perspective view of a cross bracing link.

FIG. 49 is a perspective view of a preferred embodiment of a multi-level selective axis spinal plating fixation system.

FIG. 50 is a perspective view of a preferred embodiment of another multi-level selective axis spinal plating fixation system.

FIG. 51 is a perspective view of a preferred embodiment of a pair of multi-level selective axis spinal plating fixation systems including a cross-brace.

### DETAILED DESCRIPTION

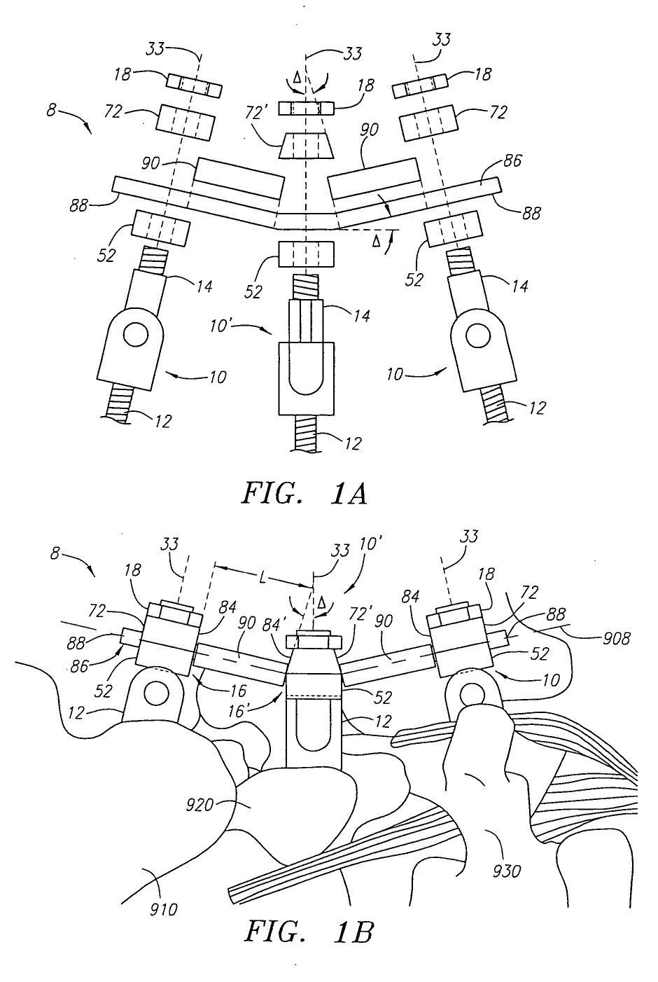

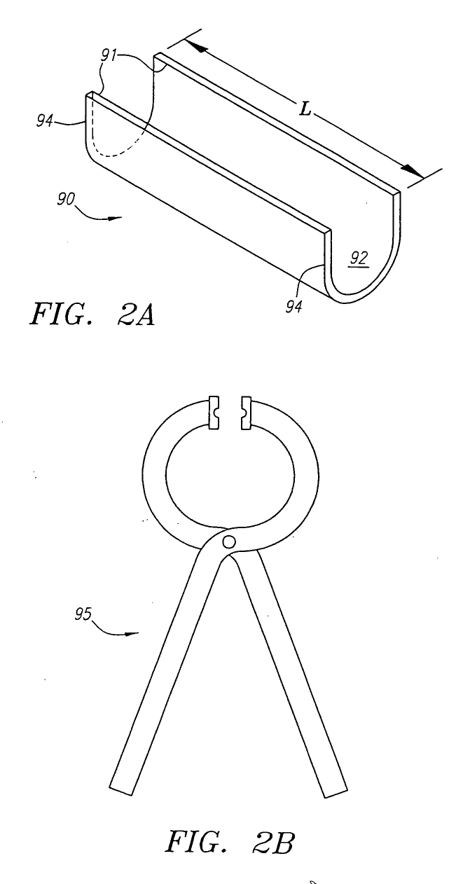

Turning now to the drawings, FIGS. 1A and 1B show a preferred embodiment of a spinal fixation system 8, in accordance with the present invention. Generally, the system 8 includes a plurality of anchor screw assemblies, 10, a rod 86 securable between the anchor screw assemblies 10, and one or more spacer clips 90 securable to the rod 86. All of the components of the system 8 may be made from a variety of biocompatible materials, e.g., metals, and preferably from titanium or alloys including titanium.

The rod 86 may be a substantially rigid elongate member, e.g., a solid rod, having a generally round cross-section. Optionally, the rod 86 has one or more flattened regions (not shown) extending between ends 88 of the rod 86. For example, the rod 86 may include opposing flattened regions (not shown), thereby defining a flattened elliptical cross-section. Optionally, the rod 86 may include serrations or teeth (not shown) extending between the ends 88, which may facilitate securing the rod 86 to the anchor screw assemblies 10.

The rod 86 may be substantially straight initially (not shown), and may be bent and/or curved during a procedure, e.g., to conform to the natural curvature or lordosis of the anatomy encountered, as shown in FIG. 1A, and as described further below. The rod 86 is sufficiently rigid, however, such that, once bent, the rod 86 may substantially retain its bent shape when subjected to forces experienced during normal activity of a patient, as will be appreciated by those skilled in the art.

Turning to FIG. 2A, a spacer clip 90 is shown that may be secured to the rod 86. Generally, the clip 90 is a “C” shaped member including opposing edges 91 defining a pocket 92 that extends between ends 94 of the clip 90. The pocket 92 has a cross-section that is larger than the rod 86 such that the clip 90 may be received at least partially around the rod 86. The clip 90 may be malleable such that a tool, such as the crimping tool 95 shown in FIG. 2B, may be used to crimp or otherwise compress at least portions of the opposing edges 91 around the rod 86 after the clip 90 has been placed on the rod 86 to secure the clip 90 to the rod 86.

A variety of clips may be provided, e.g., having standard lengths “L” and/or cross-sections. For example, a set of clips (not shown) may be provided that have lengths from about one to about three centimeters (1-3 cm), e.g., at 0.125 centimeter intervals. The ends 94 of the clip 90 are preferably substantially square, although alternatively, they may be tapered, e.g., such that the length of the clip as measured along the edges 91 is greater than the length as measured from the bottom of the pocket 92 (not shown).

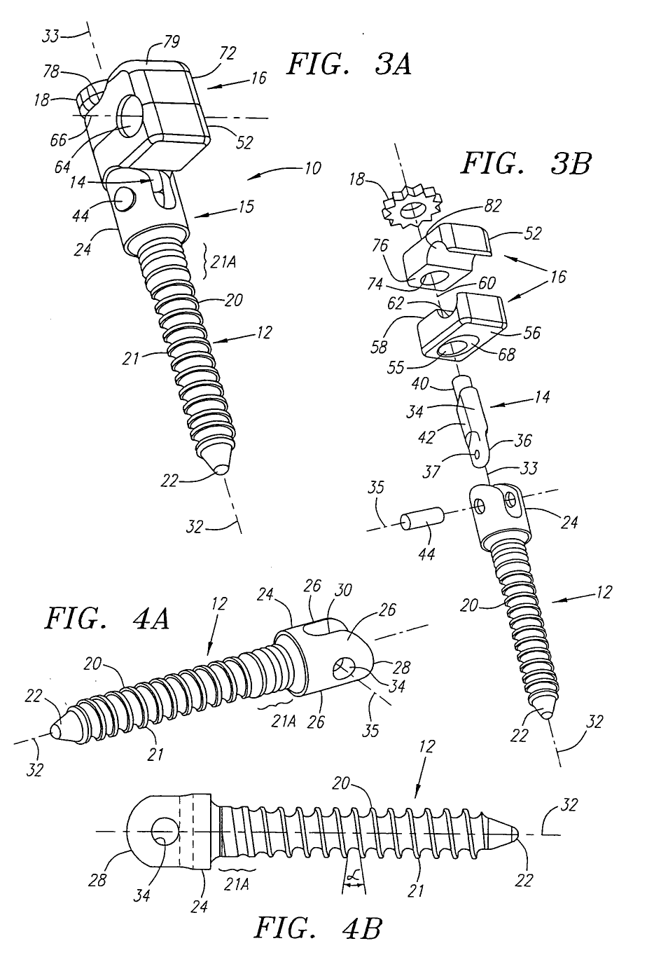

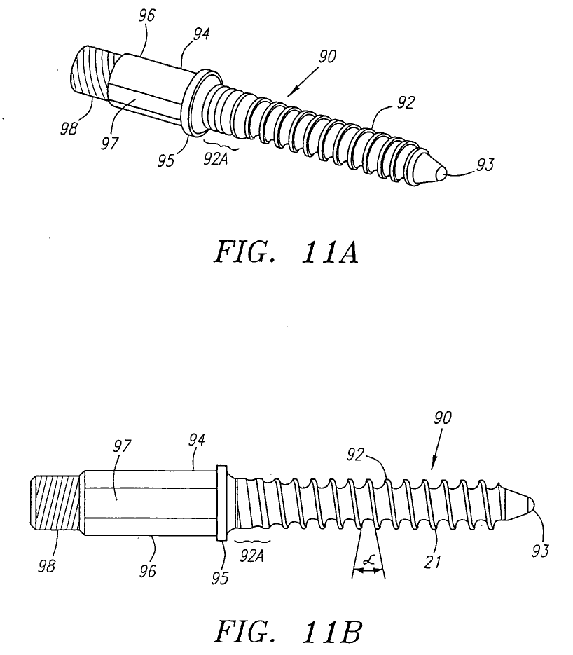

Turning to FIGS. 3-8, each of the anchor screw assemblies 10 includes a screw 12, a swing bolt 14 pivotally coupled to the screw 12 to provide an anchor screw 15, and a clamp assembly 16 securably received on the swing bolt 14, as shown in FIGS. 3A and 3B. Alternatively, other anchor screw assemblies may be used to provide a system in accordance with the present invention. For example, a rigid anchor screw 90, such as that shown in FIGS. 11A and 11B, may be used instead of an anchor screw 15 including a swing bolt 14. In a further alternative, other anchor screw assemblies may be used, e.g., including saddle or clamp assemblies mounted on screw bolts, such as those disclosed in U.S. Pat. Nos. 4,653,481, 5,487,744, and 5,545,166, the disclosures of which are expressly incorporated herein by reference. Thus, any known anchor screw assemblies may be used, although the anchor screw assemblies shown and described herein may be particularly advantageous.

Turning to FIGS. 4A and 4B, the screw 12 of the anchor screw 15 generally includes a first threaded portion 20 terminating in a tip 22, and a second head portion 24 opposite the tip 22. The threaded portion 20 may include a helical thread 21 defining a thread pattern, preferably configured for substantially securing the screw 12 into bone, such as a portion of a vertebra (not shown). The thread spacing may be between about three to six threads per centimeter (3-6 threads/cm), and preferably about 4.8 threads per centimeter (about 12 threads per inch). The thread spacing may be substantially constant between the tip 22 and the head portion 24 or may vary along the length of the threaded portion 20.

The leading and trailing edges of axially adjacent portions of the thread 21 may define an inclusive angle “α” between them of between about twenty to forty degrees (20-40°), and preferably about thirty degrees (30°). Preferably, each thread 21 is rounded or tapers outwardly from the root diameter to the major diameter of the thread 21, such that the leading and trailing edges on either side of a portion of the thread 21 define tangent lines that intersect one another adjacent the outer edge of the respective portion of the thread 21. The thread 21 may have a height of between about 0.50-3.00 millimeters, and preferably between about 0.60-2.00 millimeters.

The threaded portion 20 may have desired dimensions to accommodate threading into bone, such as a vertebra (not shown). For example, the threaded portion 20 may have an outer diameter between about 3.5-8.5 millimeters, preferably between about 5.8-8.5 millimeters, and a length between about 25-65 millimeters, and preferably between about 35-65 millimeters. The threaded portion 20 may have a substantially uniform major and minor diameter along its length. Alternatively, the threaded portion 20 may have a taper, e.g., reducing in minor and/or major diameter from the head portion 24 towards the tip 22. The thread 21 may have a substantially uniform height, or may become increasingly higher from the head portion 24 towards the tip 22, e.g., if the threaded portion 20 is tapered, to provide a substantially uniform outer diameter for the threaded portion 20.

In addition, the threaded portion 20 may include a pull-out portion 21A, which may facilitate manufacturing of the anchor screw 12 and/or may improve engagement of the screw 12 with bone into which the screw 12 is threaded. Other thread patterns and screw designs may also be used for an anchor screw assembly in accordance with the present invention, as found in U.S. Pat. Nos. 4,854,311, 5,034,011, and 5,226,766, the disclosures of which are expressly incorporated herein by reference.

The head portion 24 generally has a cross-section larger than the threaded portion 20 and includes a full-radius shoulder 28 opposite the threaded portion 20. The shoulder 28 includes a predetermined radius about a pivot axis 35 to facilitate pivoting of the swing bolt 14 and/or the clamp assembly 16 (shown in FIGS. 3A and 3B) with respect to the head portion 24, as explained further below. The head portion 24 includes a slot 30 therein extending generally parallel to a longitudinal axis 32 of the screw 12, thereby dividing the head portion 24 into ears 26. Pin holes 34 extend through the ears 26 along the pivot axis 35, i.e., substantially perpendicular to the longitudinal axis 32.

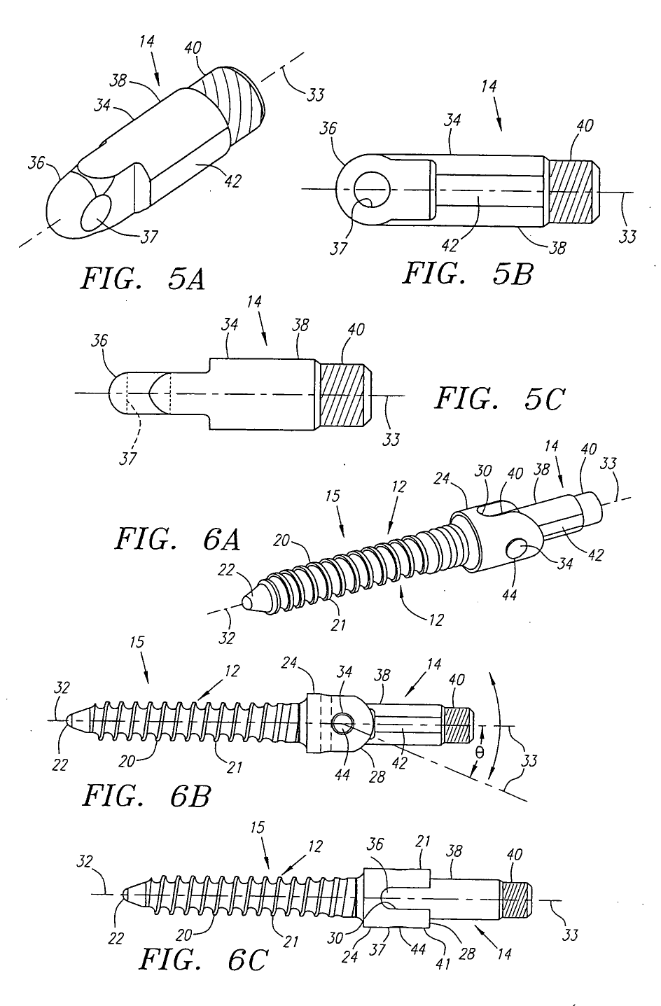

Turning to FIGS. 5A-5C, the swing bolt 14 includes an elongate body 34 including a first looped region 36, a second noncircular intermediate region 38, and a third threaded region 40 generally opposite the looped region 36. The looped region 36 may be substantially narrower than the other regions of the swing bolt 14, i.e., having a width slightly smaller than a width of the slot 30 in the screw 12 such that the looped region 36 may be received in the slot 30 between the ears 26, as shown in FIGS. 6A-6C. The looped region 36 has a pin hole 37 therethrough that extends substantially perpendicular to the longitudinal axis 32.

The noncircular region 38 of the swing bolt 14 is preferably substantially smooth-walled and has a noncircular cross-section, preferably for slidably receiving the clamp assembly 16 thereon (as shown in FIGS. 3A and 3B), while preventing rotation of the clamp assembly 16 about longitudinal axis 33. In the embodiment shown in FIGS. 5A-5C, one or more flat walls 42, and preferably two opposing flat walls, are formed along the intermediate region 38. Thus, the cross-section may define a flattened elliptical shape, a “D” shape. Alternatively, other shapes may be used, such as a hexagon, a square, a star, or other noncircular geometric shape.

As shown in FIGS. 6A-6C, the looped region 36 of the swing bolt 14 may be received in the slot 30 of the head portion 24, and a pin 44 may be received through the pin holes 34, 37 to provide anchor screw 15. The pin 44 may fix the swing bolt 14 to the screw 12, while allowing the swing bolt 14 and screw 12 to pivot with respect to one another such that the longitudinal axes 32, 33 intersect, but define an angle “theta” greater than zero degrees, as shown in phantom in FIG. 6B.

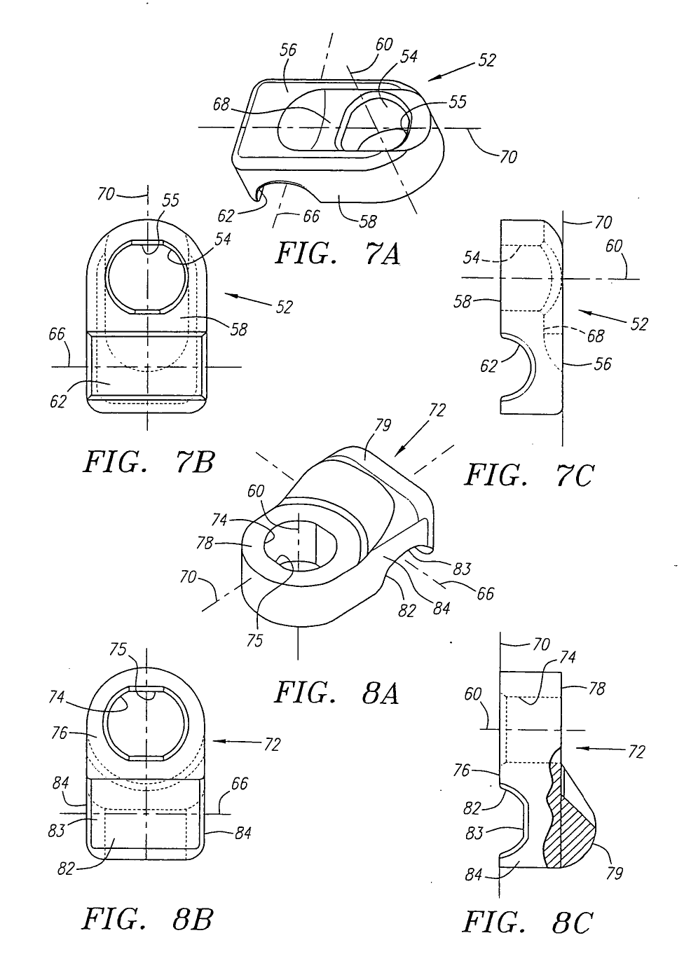

Turning to FIGS. 7A-8C, the clamp assembly 16 (shown in FIGS. 3A and 3B) generally includes a first lower clamp portion 52 and a second upper clamp portion 72. The lower and upper clamp portions 52, 72 have noncircular bolt passages 54, 74 that extend entirely through them between lower surfaces 56, 76 and upper surfaces 58, 78, respectively, thereby defining a first axis 60. The bolt passages 54, 74 preferably have a cross-section similar to the cross-section of the noncircular region 42 of the swing bolt 14 (see FIGS. 3B, 5A, 5B, 6A, and 6B). Thus, the bolt passages 54, 74 may accommodate receiving the swing bolt 14 therethrough, while preventing rotation of the clamp assembly 16 on the swing bolt 14, as explained further below.

In addition, the lower and upper clamp portions 52, 72 have generally semi-cylindrical grooves 62, 82 therein that cooperate with one another when the clamp assembly 16 is assembled to define a rod passage 64, as shown in FIG. 3A. The rod passage 64 generally extends along a second axis 66 that is transverse to, and preferably substantially perpendicular to, the first axis 60. In the embodiment shown, the second axis 66 is also substantially perpendicular to a third axis 70 that extends along a length of the lower clamp portion 52 substantially perpendicular to both the first and second axes 60, 66 (thus, the three axes 60, 66, 70 may be orthogonal to one another). The rod passage 64 has a cross-section similar to a rod 86 (not shown, see FIG. 1A) that may be received therein. For example, the cross-section may be generally circular, but optionally may be noncircular, e.g., circular with one or more flattened walls, such as wall 83 shown in the upper clamp portion 72 in FIGS. 8B and 8C. Alternatively, as shown in FIG. 3A, the rod passage 64 may have other geometric shapes, similar to the bolt passages 54, 74, described above. In a further alternative, one or both of the grooves 62, 82 may include teeth or other serrations (not shown) for enhancing engagement with the rod received in the rod passage 64 (shown in FIG. 3A), either alone or in combination with one of the cross-sections described above. Exemplary serrations are shown in U.S. Pat. Nos. 4,653,481 and 5,545,164, the disclosures of which are expressly incorporated herein by reference.

With particular reference to FIGS. 8A-8C, the groove 82 in the upper clamp portion 72 extends along the lower surface 76. The upper surface 78 may be recessed around the bolt passage 74, thereby accommodating a fastener (not shown) thereon, while minimizing the profile of the resulting clamp assembly. For example, the groove 82 may define a hump 79 opposite the lower surface 76, which may be higher than the upper surface 78. The hump 79 may have a height similar to a nut or other fastener (not shown) that may be attached to a swing bolt (also not shown) that is inserted through the bolt passage 74. Thus, when a fastener 18 (not shown, see FIG. 3A) engages the upper surface 78, the upper surface of the fastener 18 may define a height similar to the hump 79 (also as shown in FIG. 3A), thereby substantially minimizing a profile of the anchor screw assembly and/or reducing tissue irritation.

Returning to FIGS. 8A-8C, side edges 84 of the upper clamp portion may be substantially square with the lower surface 76, i.e., the side edges 84 may extend substantially parallel to the first axis 60. Alternatively, as shown in FIGS. 15A and 15B, the side edges 84′ may be tapered. For example, the side edges 84′ of the upper clamp portion 72′ may taper inwardly towards the hump 79′ such that they define an angle Δ between about one and twenty degrees (1-20°) with the first axis 60,′ and preferably about ten degrees (10°). Such a taper may be machined into the side edges 84′ or formed using other known methods.

Alternatively, as shown in FIG. 15C, instead of tapering the side edges 84, the side edges 84 may be substantially square, and a spot face, recess, or notch 85″ may be formed around the groove 82,″ e.g., by spot facing the side edges 84.″ These tapered or recessed features may enhance abutment with ends 94 of the spacer clip 90 despite the natural curvature or lordosis of a spinal column (not shown) when the spacer clip 90 is secured to a curved rod 86 (not shown, see FIGS. 1A and 1B), as explained further below.

Turning to FIGS. 7A-7C, the groove 62 in the lower clamp portion 52 extends along the upper surface 58. In addition, the lower clamp portion 62 also includes a recess 68 in the lower surface 56 that intersects the bolt passage 54. The recess 68 preferably has a radius of curvature similar to the shoulder 28 on the head portion 24 of the screw 12 (see FIGS. 6A-6C), as explained further below.

Returning to FIGS. 3A, 3B, 7C, and 8C, the lower surface 76 of the upper clamp portion 72 and the upper surface 58 of the lower clamp portion 52 are substantially flat such that the lower and upper clamp portions 52, 72 may substantially abut one another to provide the rod passage 64. Alternatively, the upper and lower surfaces 58, 76 may include mating segments, e.g., cooperating tabs and slots or other male/female connectors (not shown), that may positively engage one another when the lower and upper clamp portions 52, 72 are disposed in the proper orientation.

The clamp assembly 16 may be received on the swing bolt 14, e.g., by orienting the clamp assembly 16 such that the bolt passages 54, 74 are properly aligned with the noncircular region of the swing bolt 14. The lower clamp portion 52 may be directed over the swing bolt 14 and then the upper clamp portion 72 may be received over the swing bolt 14, i.e., through the bolt passages 54, 74, respectively. A fastener, e.g., nut 18, may be threaded onto the threaded region 40 of the swing bolt 14 until it engages upper surface 78 of the upper clamp portion 72, thereby forcing the clamp assembly 16 towards the head portion 24 of the screw 12. Consequently, the lower clamp portion 52 may abut the head portion 24 such that the shoulder 28 is received in the recess 68 in the lower surface 56.

Preferably, because of the mating shapes of the shoulder 28 and recess 68, the lower clamp portion 52 may slide along the shoulder 28 as the swing bolt 14 is pivoted with respect to the screw 12. Once a desired angle is obtained, the nut 18 may be further tightened until the wall of the recess 68 frictionally engages the shoulder 28, thereby substantially securing the swing bolt 14 at the desired angle relative to the screw 12.

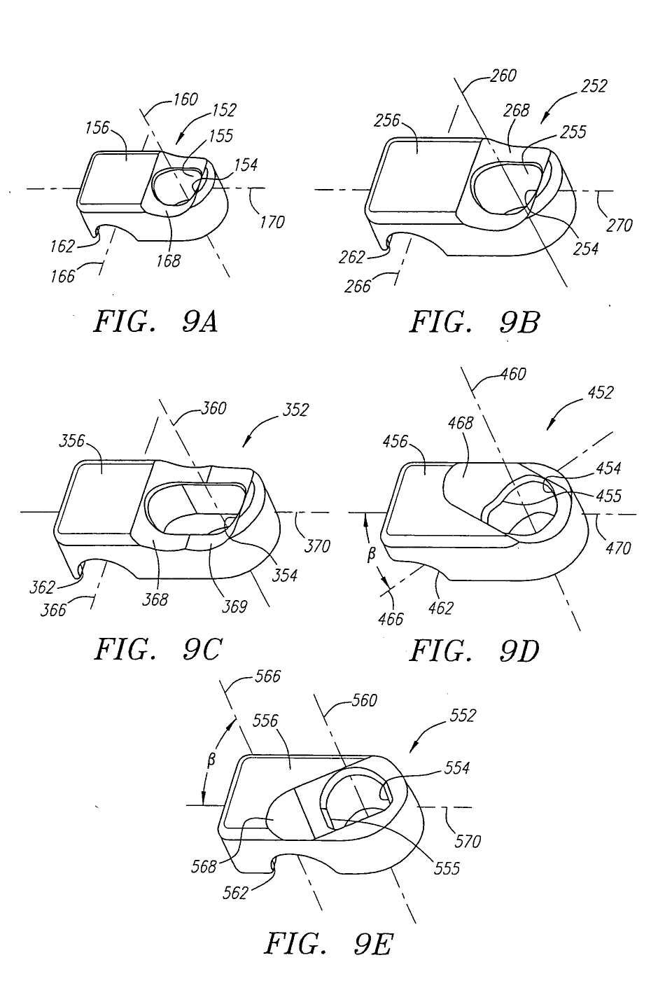

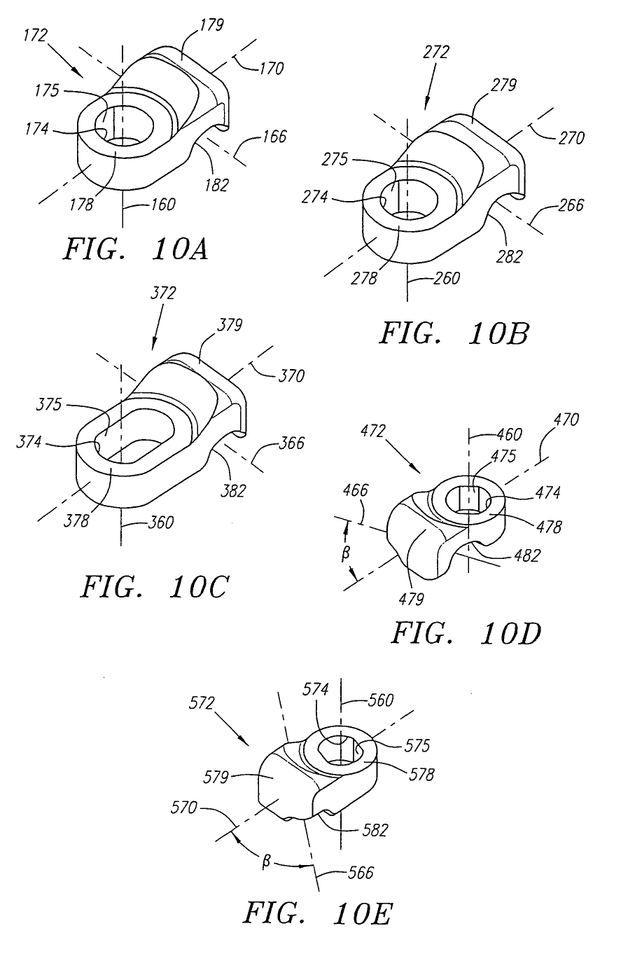

Turning to FIGS. 9A-10E, several alternative embodiments of lower and upper clamp portions are shown that together may provide clamp assemblies that may be received over the screw assembly 15 of FIGS. 6A-6C. For example, the lower and upper clamp portions 152, 172 shown in FIGS. 9A and 10A are generally similar to that shown in FIGS. 7A and 8A, except that the flat regions 155, 175 of the bolt passages 154, 174 and the recess 168 are offset ninety degrees from the previous embodiment. The resulting clamp assembly (not shown) may be mounted similar to the previous embodiment, but offset ninety degrees with respect to the anchor screw (not shown).

Turning to FIGS. 9B and 10B, another set of lower and upper clamp portions 252, 272 are shown that are similar to the embodiments of FIGS. 9A and 10A, except that the grooves 262, 282 are located further away from the bolt passages 254, 274 along the third axis 270. The resulting clamp assembly from these embodiments may be mounted on the anchor screw similar to the previous embodiment with the shoulder 28 of the screw 12 being received in the recess 268. A rod (not shown) received in the resulting rod passage, however, will be disposed further from the anchor screw than the previous embodiment.

Turning to FIGS. 9C and 10C, yet another set of lower and upper clamp portions 352, 372 are shown that are similar to the embodiments of FIGS. 9A and 10A, except that the bolt passages 354, 374 have an elongated elliptical shape extending along the third axis 370. In addition, the lower surface 356 of the lower clamp portion 352 includes adjacent recesses 368, 369 that intersect the bolt passage 354 and may overlap one another. The resulting clamp assembly from this embodiment may be secured to the anchor screw such that either of the recesses 368, 369 slidably engages the shoulder of the screw (not shown), thereby allowing a rod (also not shown) received in the rod passage to be disposed at two possible locations, e.g., distances, relative to the anchor screw. Optionally, more than two recesses (not shown) may be provided, thereby allowing the rod passage to be disposed at multiple distances from the anchor screw.

Turning to FIGS. 9D and 10D, still another set of lower and upper clamp portions 452, 472 are shown that are similar to the embodiments of FIGS. 7A and 8A, except that the grooves 462, 482 and recess 468 are aligned such that the second axis 466 defines an angle “β∞ with the third axis 470. Preferably, the angle “β” is between about ten and seventy degrees (10-75°), and more preferably between about thirty and forty five degrees (30-45°). In addition, the flattened wall regions 455, 475 are aligned substantially parallel to the second axis 466, thereby also defining an angle “β” with respect to the third axis 470.

Turning to FIGS. 9E and 10E, another set of lower and upper clamp portions 552, 572 are shown that are similar to the embodiments of FIGS. 9D and 10D, except that the bolt passages 554, 574, recess 568, and grooves 562, 582 are mirror opposites or opposite-hand of those in the previous embodiment. Thus, it will be appreciated by those skilled in the art that a variety of clamp assemblies may be providing including a range of dimensions, e.g., lengths, thicknesses, “β” angles, and the like, in any combination or subcombination of the features described above.

Turning to FIGS. 11A and 11B, another embodiment of an anchor screw 90 is shown that includes a threaded portion 92 terminating in a tip 93, and an enlarged head portion 94 including a noncircular region 96 and a threaded region 98 opposite the tip 93. The threaded portion 92 may include any of the features and/or dimensions described above for the anchor screw 12 of FIGS. 4A-4C, e.g., thread pattern, outer diameter, taper, and the like. The threaded region 98 may receive a fastener, such as the nut described above (not shown), e.g., to substantially secure a clamp assembly (also not shown) on the noncircular region 96, similar to the embodiment described above. Thus, the anchor screw 90 may receive any of the clamp assemblies described above.

To select a system 8, such as that shown in FIGS. 1A and 1B, a kit may be provided (not shown). The kit may include a plurality of anchor screws, clamp assemblies, and fasteners that may be selected based upon the specific vertebrae being treated and/or based upon the anatomy encountered. Each anchor screw assembly may include an anchor screw, e.g., a pivoting or fixed anchor screw, and one or more clamp assemblies. For example, a plurality of upper and lower clamp assemblies may be provided, having different dimensions, as described above. An appropriate pair, corresponding to the patient anatomy encountered, may be selected for each anchor screw.

One or more rods may be provided, and an apparatus (not shown) may be provided for bending the rod(s) in a desired configuration during a procedure. Finally, a plurality of spacer clips may be provided, e.g., having different lengths, as described above, and a tool, e.g., a crimper or pliers, may be provided for crimping the spacer clip. Thus, a system in accordance with the present invention provides a modularity that may easily accommodate a variety of anatomy and patients.

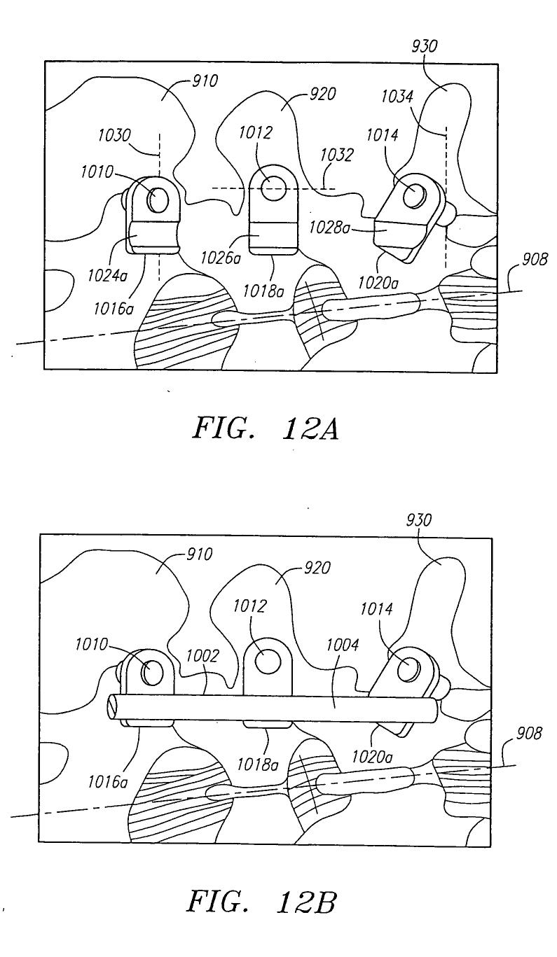

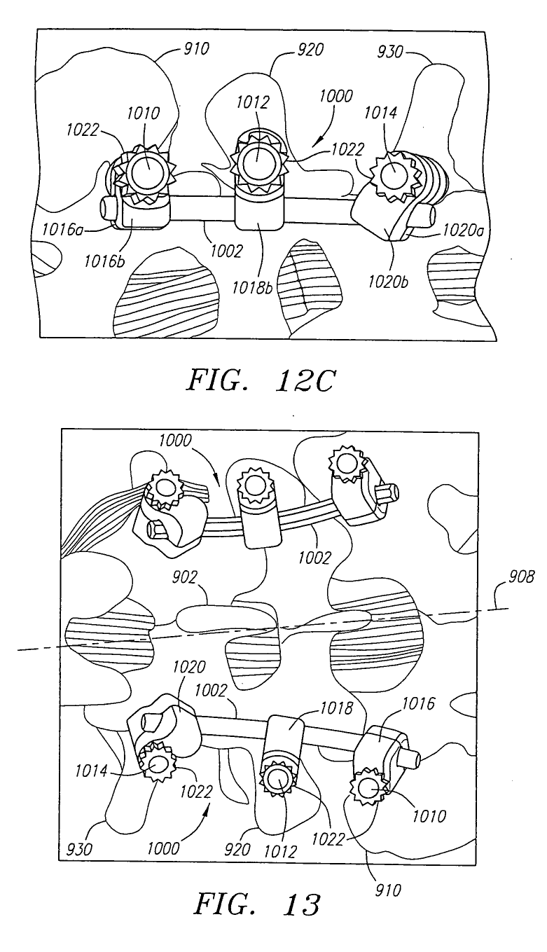

Turning to FIGS. 12A-12C and 13, an exemplary system 1000 is shown that includes a pair of rods 1002 that are each implanted along a spinal column using three swing bolt anchor screws 1010-1014 and three clamp assemblies, 1016-1020. Alternatively, one or more of the swing bolt anchor screws, such as the outside anchor screws 1010, 1014, may be replaced with nonpivoting anchor screws (such as that shown in FIGS. 11A and 11B). In a further alternative, fewer or additional anchor screws may be implanted, e.g., to secure a shorter or longer rod and/or to fix fewer or additional vertebrae.

Preferably, the rods 1002 are implanted generally parallel to the central spinal axis on either side of the spinous processes 902, as shown in FIG. 13. The system 1000 may be used to provide adjustment of the vertebrae, e.g., to allow vertical or horizontal, medial or lateral adjustment. Although an implantation procedure for only one rod 1002 is described below, it will be appreciated that a second rod (or even additional rods) may be implanted using a similar procedure.

Turning first to FIG. 12A, the vertebrae, e.g., vertebrae 910, 920, 930, to be stabilized are exposed, e.g., using conventional surgical procedures. The anchor screws 1010-1014 are screwed into the vertebrae 910-930, respectively, e.g., into the pedicles. Preferably, the anchor screws 1010-1014 are screwed in sufficiently to provide a predetermined pivot axis with respect to a centerline spinal axis of the patient. For example, the anchor screw 1012 may be screwed into the pedicle until a pivot axis 1032 of the anchor screw 1012 is disposed generally parallel to the centerline spinal axis 908. In contrast, the other anchor screws 1010, 1014 may be screwed into their respective vertebrae until their respective pivot axes 1030, 1034 are disposed substantially transverse to the first pivot axis 1032, and preferably substantially perpendicular to the centerline spinal axis.

Clamp assemblies 1016-1020 are selected based upon the anatomy encountered. For example, the clamp assembly 1016 may be similar to the clamp assembly 52, 72 shown in FIGS. 7A and 8A, and the clamp assembly 1018 may be similar to the clamp assembly 152, 172 of FIGS. 9A and 10A, i.e., having a longer length than the clamp assembly 1016. Finally, the clamp assembly 1020 may be similar to the clamp assembly 452, 472 shown in FIGS. 9B and 10B, i.e., having a groove 1028a (see FIG. 12A) that extends in line with the grooves 1024a, 1026a of the clamp assemblies 1016, 1018.

The lower clamp portions 1016a-1020a of the clamp assemblies 1016-1020 may be received over the noncircular regions (not shown) of the anchor screws 1010-1014, as best seen in FIG. 12A.

A rod 1002 may be received in the grooves 1024a-1028a in the lower clamp portions 1016a-1020a, thereby extending between the anchor screws 1010-1014, as shown in FIG. 12B. If desired, the rod 1002 may be bent to a predetermined shape, as needed, to conform to the anatomy alignment encountered. Preferably, the rod 1002 is bent in only one plane, e.g., the sagetal plane, while remaining substantially straight in the coronal plane, as shown in FIG. 1A. “Sagetal” plane, as used herein, refers to the plane that may be seen from a lateral view of the patient, e.g., that is viewed horizontally when the patient is lying face-down (such as the plane seen in FIGS. 1A and 1B). “Coronal” plane refers to the plane that may be seen from an anterior or posterior view of the patient, e.g., that is viewed vertically up the length of the spine when the patient is lying face-down (such as that shown in FIGS. 12A-12C).

Optionally, if the rod 1002 includes one or more flattened regions 1004, the flattened region(s) 1004 may be oriented so that they may engage similar flattened regions (not shown) in the rod passages 1024-1028 in the clamp assemblies 1016-1020 (e.g., in the upper clamp portions 1016b-1020b).

One or more of the clamp assemblies 1016-1020 may be adjusted at any time during the procedure. By adjusting the clamp assemblies 1016-1020, the swing bolts on the anchor screws 1010-1014 may be pivoted about their respective pivot axes 1030-1034 with respect to the threaded portions that have been threaded into the vertebrae 910-930. For example, the lower clamp portions 1016a-1020a may be adjusted before and/or after the rod 1002 is received in the grooves 1024a-1028a. Because the pivot axes 1030-1034 of the swing bolt anchor screws 1010-1014 are substantially transverse with respect to one another, a uniaxial device (i.e., pivoting in a single axis) may be used to provide multiple degrees of freedom for moving the clamp assemblies 1016-1020 relative to the rod 1002. This may minimize the amount of bending required of the rod 1002, preferably requiring bending in only one plane (preferably, the sagetal plane), thereby substantially maximizing the rigidity of the rod 1002.

As shown in FIG. 12C, upper clamp portions 1016b-1020b may be placed on the lower clamp portions 1016a-1020a, i.e., received on the swing bolts of the anchor screws 1010-1014. When properly placed, the grooves (not shown) in the upper clamp portions 1016b-1020b substantially engage the rod 1002. Fasteners, such as nuts 1022, may then by threaded onto the swing bolts, thereby substantially securing the rod 1002 between the upper and lower clamp portions 1016-1020.

Preferably, the nuts 1022 are twelve point jam nuts. The nuts 1022 may have rounded upper edges, which may minimize tissue irritation, e.g., of tissue overlying the nuts 1022 after implantation of the system 1000. In addition, the nuts 1022 may include a crimpable rim (not shown), which may be crimped when the nuts are tightened to a desired torque, e.g., to prevent subsequent loosening of the nuts. Alternatively, hex nuts or other fasteners may be used.

Preferably, the lower clamp portions 1016a-1020a include radiused recesses (not shown) on their lower surfaces that intersect bolt passages for receiving the swing bolts 1010-1014 therein. These recesses may slidably engage similarly radiused shoulders on screws of the swing bolts (not shown), as described above. Thus, as the angles of the swing bolts are adjusted, the shoulders may pivotally slide along the surfaces of the recesses of the lower clamp portions 1016a-1020a. Once a desired configuration is obtained, the nuts 1022 may be tightened, thereby causing the lower clamp portions 1016a-1020a to frictionally engage the shoulders and secure the swing bolts with respect to the threaded portions without substantially moving one or more of the vertebrae out of the desired position.

If it is desired to adjust the vertebrae 910-930 with respect to one another, the nuts 1022 may be loosened, and the vertebrae 910-930 adjusted, thereby possibly changing the angle of one or more of the clamp assemblies 1016-1020 holding the rod 1002. Once a desired arrangement is obtained, the nuts 1022 may be tightened, thereby securing the clamp assemblies 1016-1020. Thus, with a system in accordance with the present invention, each individual clamp assembly is uniaxial, i.e., may only be pivoted about a single axis. By setting the axes of the anchor screws substantially transverse relative to one another, substantially flexibility may be obtained without substantially compromising vertebra position. Because of the uniaxial nature of the clamp assemblies, the system may be less likely to become misaligned when the patient resumes normal activity than a polyaxial system.

Once the final configuration of the clamp assemblies 1016-1020 and/or anchor screws 1010-1014 is determined, spacer clips 90 (not shown, see FIGS. 1A and 1B) may be placed on the rod 1004 between adjacent anchor screw assemblies.

For example, turning to FIGS. 1A and 1B, an exemplary system 8 is shown that has been implanted into vertebrae 910, 920, 930. After vertebral distraction or compression and before final tightening of the nuts 18, spacer clips 90 may be placed around and crimped to exposed portions of rod 86 that extends between adjacent clamp assemblies 10, 10′.

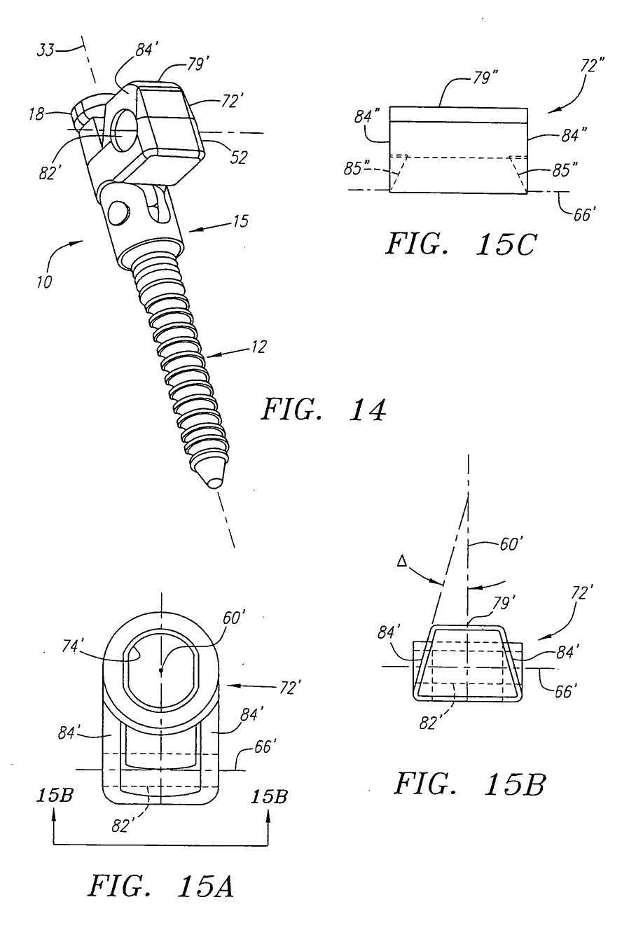

In this exemplary system 8, two anchor screw assemblies 10 including upper clamp portions 16 with square side edges 84 (such as that shown in FIG. 3A) and one anchor screw assembly 10′ (such as that shown in FIG. 14) with an upper clamp portion 16′ with tapered side edges 84′ have been selected. Because of the natural curvature or lordosis defined by the vertebrae 910, 920, 930, the longitudinal axes 33 of adjacent anchor screws 15 may define an angle Δ relative to one another. To match this natural curvature, the rod 86 may be bent to also define an angle Δ.

The upper clamp portion 16′ may be tapered, as described above with reference to FIGS. 15A and 15B, also to define an angle Δ relative to the longitudinal axis 33. With this anchor assembly 10′ selected, the opposing side edges 84, 84′ of the adjacent upper clamp portions 72, 72′ may be oriented substantially parallel to one another, as best seen in FIG. 1B. The distance “L” between the opposing side edges 84, 84′ may be measured, and an appropriate spacer clip 90, e.g., having a length approximately “L” (or less than “L”), i.e., corresponding substantially to the measured distance, may be placed on the exposed portion of the rod 86 between the adjacent clamp assemblies 16, 16′.

The spacer clip 90 may be crimped around the rod 86, e.g., by compressing the opposing edges 91 (not shown, see FIG. 2A) of the clip 90 using a crimper tool 95 (not shown, see FIG. 2B) to malleably deform the opposing edges 91 towards one another. The crimper tool 95 may be sufficiently long that a substantial length of the clip 90 may be engaged therein. Alternatively, one or more successive portions along the length of the clip 90 may be crimped around the rod 86 with the tool 95. This placement and crimping process may be repeated for each exposed portion of the rod(s) 90 extending between adjacent clamp assemblies 16, 16′.

The spacer clips 90 provide axial support, e.g., once the patient returns to a vertical position, thereby preventing the adjacent clamp assemblies 16, 16′ from moving substantially towards one another. Thus, the spacer clips 90 may enhance the system 8 remaining in the final configuration set during the procedure and minimize any slippage of the system 8 once the patient resumes normal activity.

In a further alternative, if the sides of the upper clamp portions are not tapered, e.g., if the upper clamp portions include spotfaced angular recesses around the rod grooves (not shown), the ends of the clip(s) may be received in the recesses, thereby preventing the clip(s) from being dislocated from the rod once the patient assumes normal activity. For example, the patient may be placed under traction or the vertebrae may otherwise be distracted away from one another. This may provide sufficient space between the adjacent clamp assemblies to allow the spacer clip(s) to be placed around and/or otherwise secured to the rod(s). When distraction is removed, the vertebrae may return to a desired state, whereupon the spacer clip(s) substantially abut the adjacent clamp assemblies, thereby preventing axial movement of the clamp assemblies towards one another. In addition, the ends of the clip(s) may be received in the recesses in the sides of the clamp assemblies, thereby further securing the clip(s) relative to the rod. In still a further alternative, the spacer clips themselves may be tapered (not shown) to extend between and abut adjacent clamp assemblies.

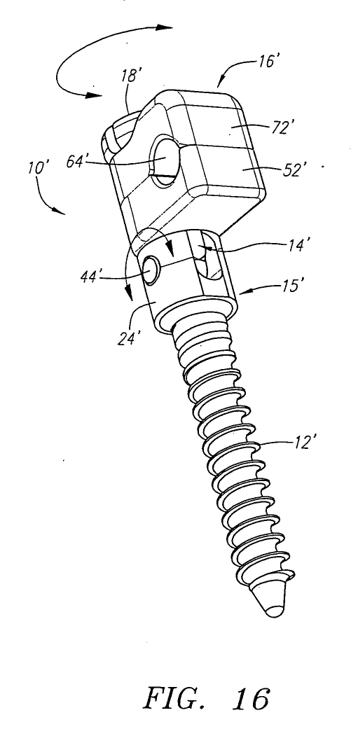

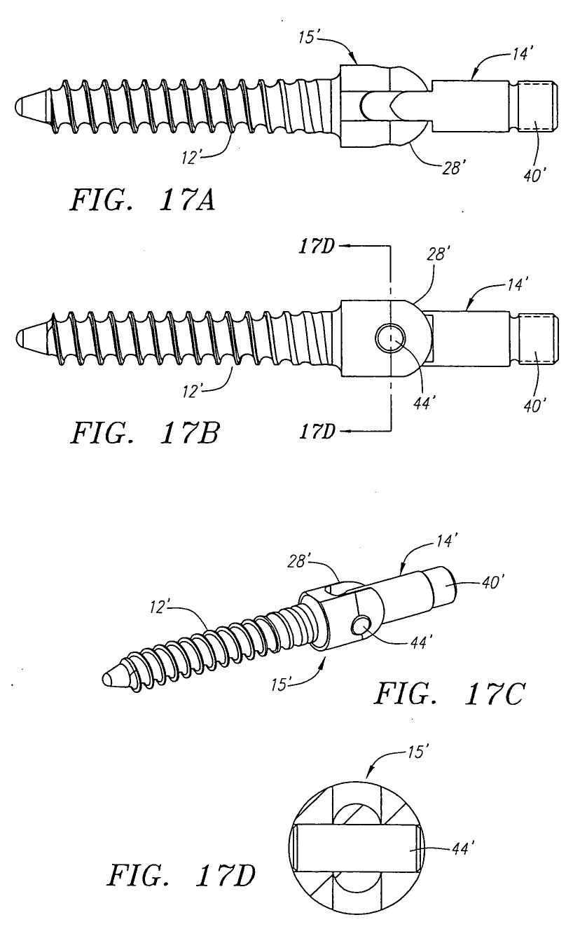

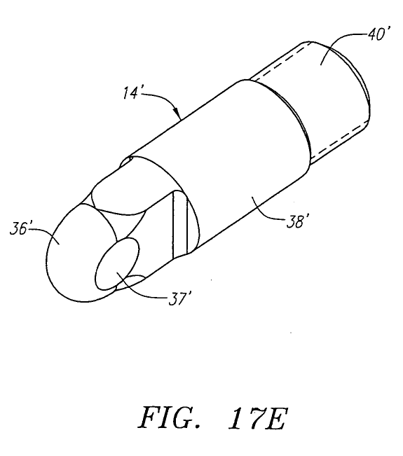

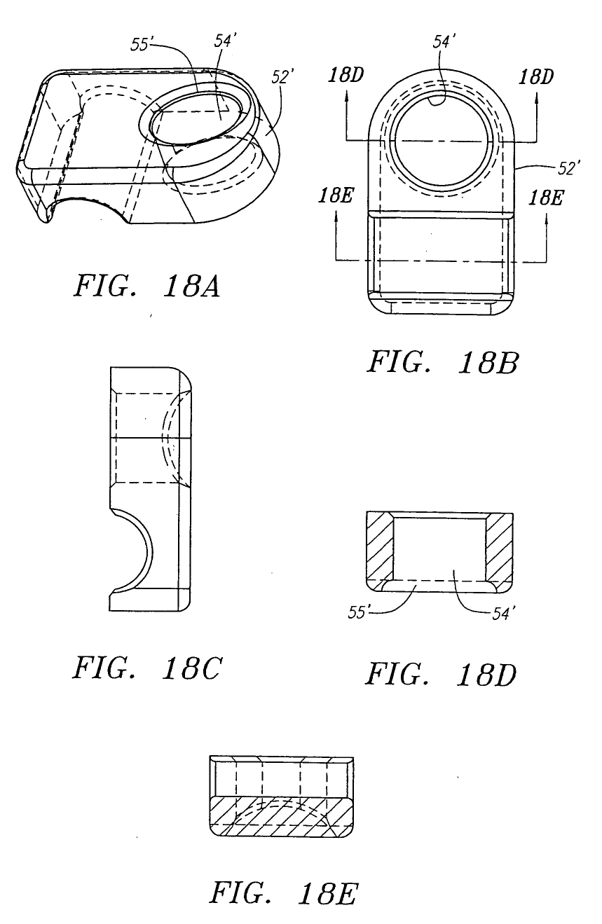

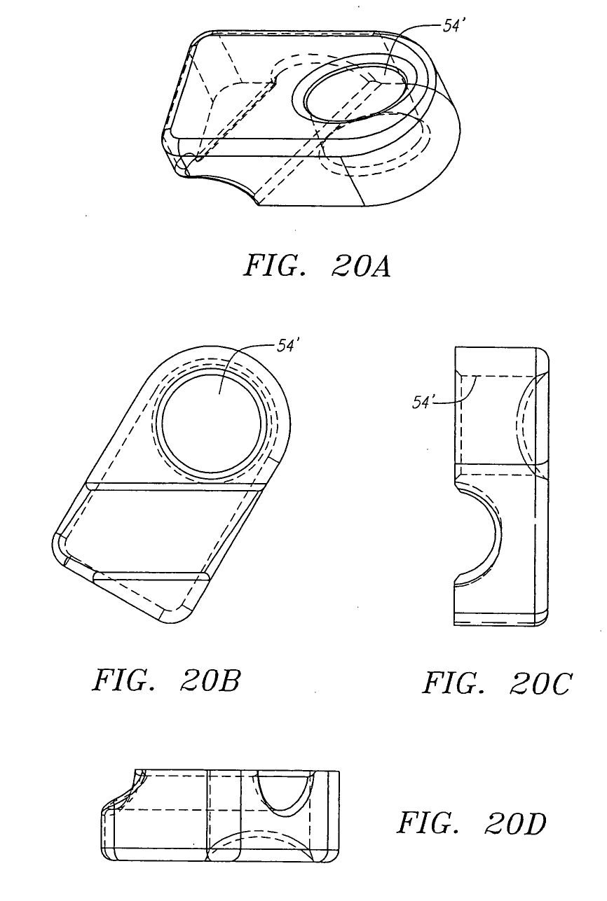

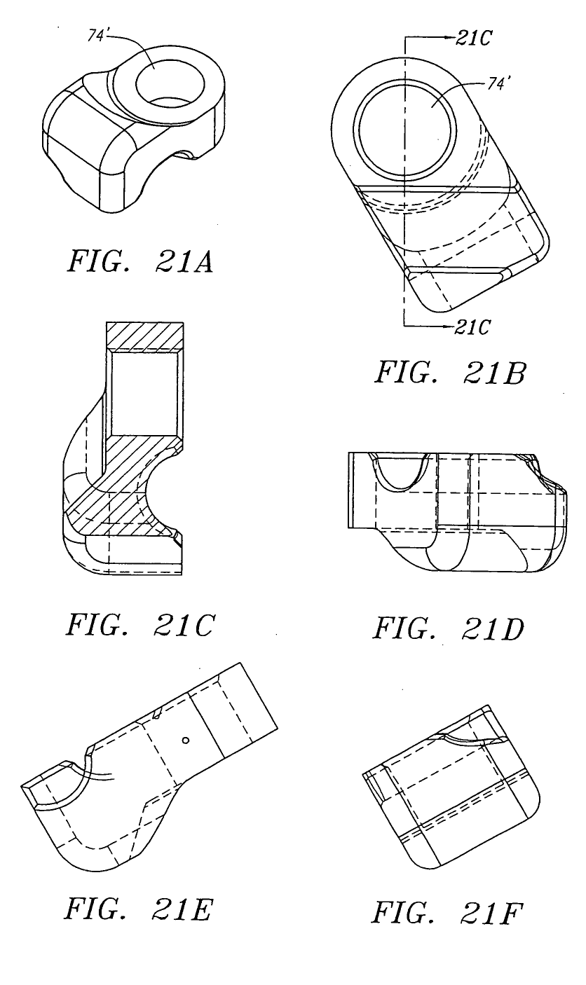

Turning now to the preferred embodiment of FIGS. 16 through 19, and the modified clamp assemblies of FIGS. 20-21 for use therewith, the same illustrate a swing anchor screw assembly 10 which is modified from the previous embodiments to provide an important multi-axial or selective axial assembly. The key structural features are the provision of a cylindrical body for the swing bolt 14′ as shown in FIG. 17e without any flat wall like 38 or 42 in FIG. 5a or other flat area or section along with a spherical shoulder 28′ on the top portion as best seen in FIG. 17a, and a mating spherical seat 55′ in the lower clamp 52′ as best seen in FIGS. 18a and 18d (as versus the radius shoulder 28 of the early embodiments). This structure allows the shoulder or upper portion 28′ to freely pivot in the seat 55′ of the lower clamp 52′ and the bolt 14′ to freely rotate in bores 54′ and 74′ within both the lower clamp 52′ and upper clamp 72′, thereby allowing any compound angle to be achieved between the screw 12′ and a rod 86 (FIG. 1) disposed in the cylindrical passageway 64′ defined between the lower 52′ and 72′ clamps.

The anchor screw 12′ itself can be constructed like those of previous embodiments, such as in FIGS. 4a-4b. Similarly, the construction of the head portion 24′ is like previous embodiments such as FIG. 4a-4b, except that it is spherical at 28′ as described above. The swing bolt 14′ is similar to previous embodiments except that it does not have a flat non-circular region such as 38 and 42 in FIGS. 5a-5c, but is completely circular. A threaded region 40′ is provided opposite the loop region 36′. The swing bolt 14 also includes a circular pin hole 37′ for receiving a pin 44′ through the head portion 24′ to allow the swing bolt 14′ to freely rotate with respect to the head portion 24′. However, the region 38′ (FIG. 17e) is completely cylindrical as previously noted and has no non-circular cross section as in earlier embodiments such as 5a-5c that would impede free rotation.

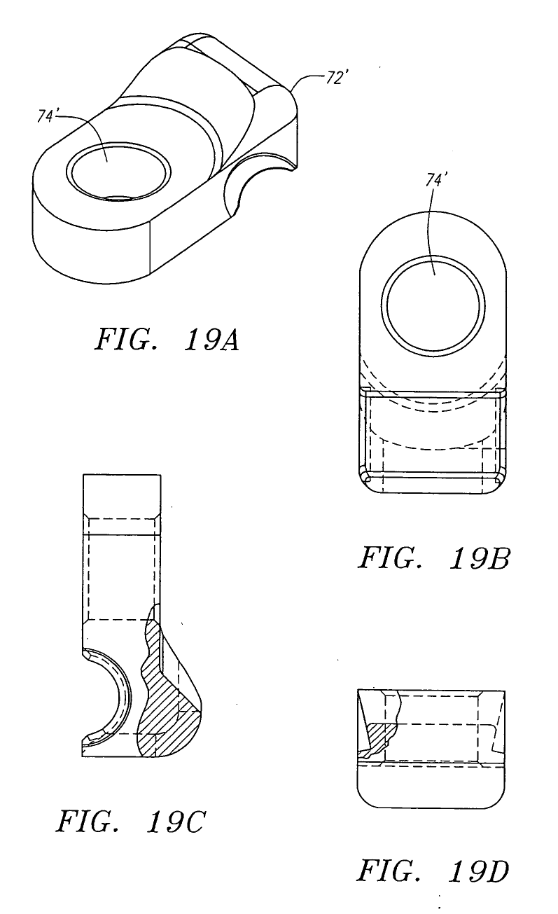

The important difference in the clamp comprising the lower clamp 52′ and upper clamp 72′ is that the openings 54′ and 74′ respectively therein for receiving the swing bolt 14′ have a cylindrical interior to mate with the cylindrical exterior region 38′ of the swing bolt 14′ to thereby allow 360° rotation of the clamp 16′ with respect to the swing bolt 14′. This is to be distinguished from early embodiments such as those shown in FIGS. 7-8 which have non-circular bolt passages 54, 74 to prevent rotation of the clamp assembly 16 on the spring bolt 14. Thus, this configuration of the lower and upper clamps in FIGS. 18-19 facilitates the multi-axial or selective axial adjustment of the clamp 16′ with respect to the anchor screw 15′. FIGS. 20 and 21 show alternative embodiments of the lower and upper clamp portions wherein the rod 86 recesses are at a different angle than those of FIGS. 18-19 and define an angle β similar to the embodiment shown in FIG. 9d. However, the clamp assembly of FIGS. 20-21 include cylindrical openings 54′ and 74′ to mate with the cylindrical section 38′ of the swing pin 14′ and allow unobstructed 360° rotation.

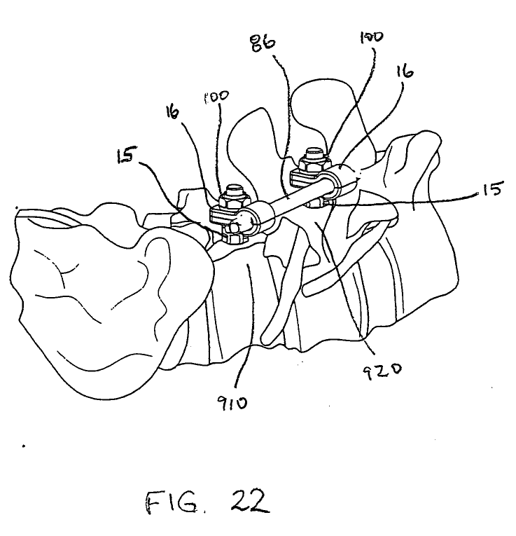

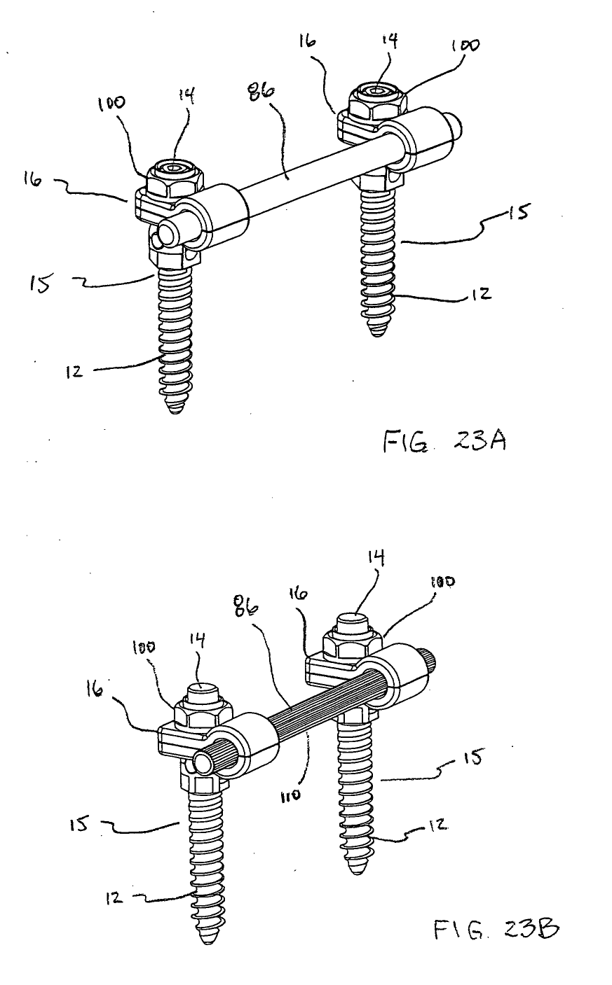

Turning now to the spinal fixation system embodiment shown in FIG. 22, the system includes a pair of anchor screws 15 attached to an adjacent pair of vertebrae 910, 920 in substantially the manner described previously herein. A rod 86 is secured between the two anchor screws 15 by a clamp assembly 16 attached to each of the anchor screws 15. A fastener secures each of the clamps 16 to the swing bolt 14 of each of the anchor screws 15. In the embodiment shown, the fasteners are jam nuts 100. As described previously, all of the components of the system are preferably made from one or more of a variety of biocompatible materials, e.g., metals, and preferably from titanium or alloys including titanium.

FIGS. 23A-B illustrate two embodiments of the present spinal fixation system. As noted above, each includes a first anchor screw 15 and a second anchor screw 15, with a rod 86 secured between the two anchor screws 15. A clamp assembly 16 is attached to the upper end of each anchor screw 15 by a jam nut 100. Each anchor screw 15 includes a screw 12 and a swing bolt 14 pivotally coupled to the screw to provide an anchor screw 15. Alternatively, other anchor screws may be used to provide a system in accordance with the present invention. For example, a rigid anchor screw 90, such as that shown in FIGS. 11A and 11B, may be used instead of an anchor screw 15 including a swing bolt 14. In still further alternative embodiments, other anchor screw assemblies may be used, e.g., including saddle or clamp assemblies mounted on screw bolts, such as those disclosed in U.S. Pat. Nos. 4,653,481, 5,487,744, and 5,545,166, the disclosures of which are expressly incorporated herein by reference. Thus, any known anchor screw assemblies may be used, although the anchor screw assemblies shown and described herein are particularly advantageous.

Turning to FIGS. 25-27, the anchor screw is preferably constructed like those of previous embodiments, such as in FIGS. 4a-4b. The head portion 24′ is like previous embodiments except that it has a spherical shoulder 28′ as described above. The spherical shoulder 28 is adapted to engage a spherical seat 55 on the clamp assembly to allow free rotation of the clamp assembly relative to the screw 12, as described below. As shown in FIG. 25, the swing bolt 14 is attached to the screw 12 by a pin 44, thereby allowing the swing bolt 14 to freely rotate with respect to the head portion 24 of the screw. The swing bolt 14, as shown in FIG. 26, is also similar to previous embodiments, having a pin hole 37 at the looped region 36, and a threaded region 40 at the end opposite the looped region. In the embodiment shown, there is no provision for a flat, non-circular region such as 38 and 42 in FIGS. 5a-5c. Instead, the intermediate region is generally cylindrical. The pin 44 is a generally cylindrical member that is sized to closely fit within the pin hole 37 on the swing bolt and the pin hole 34 on the upper end of the screw 12.

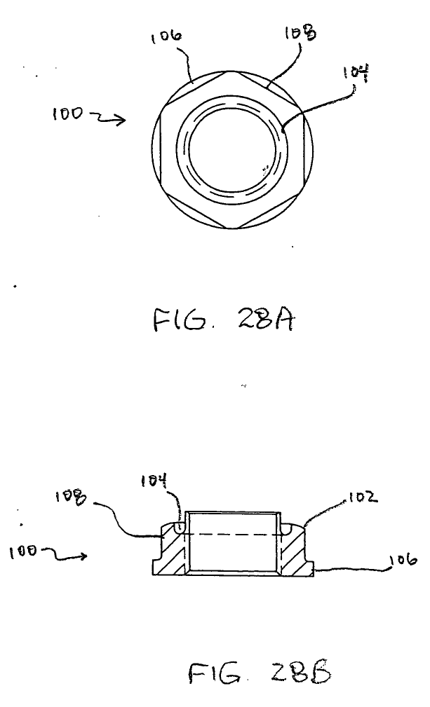

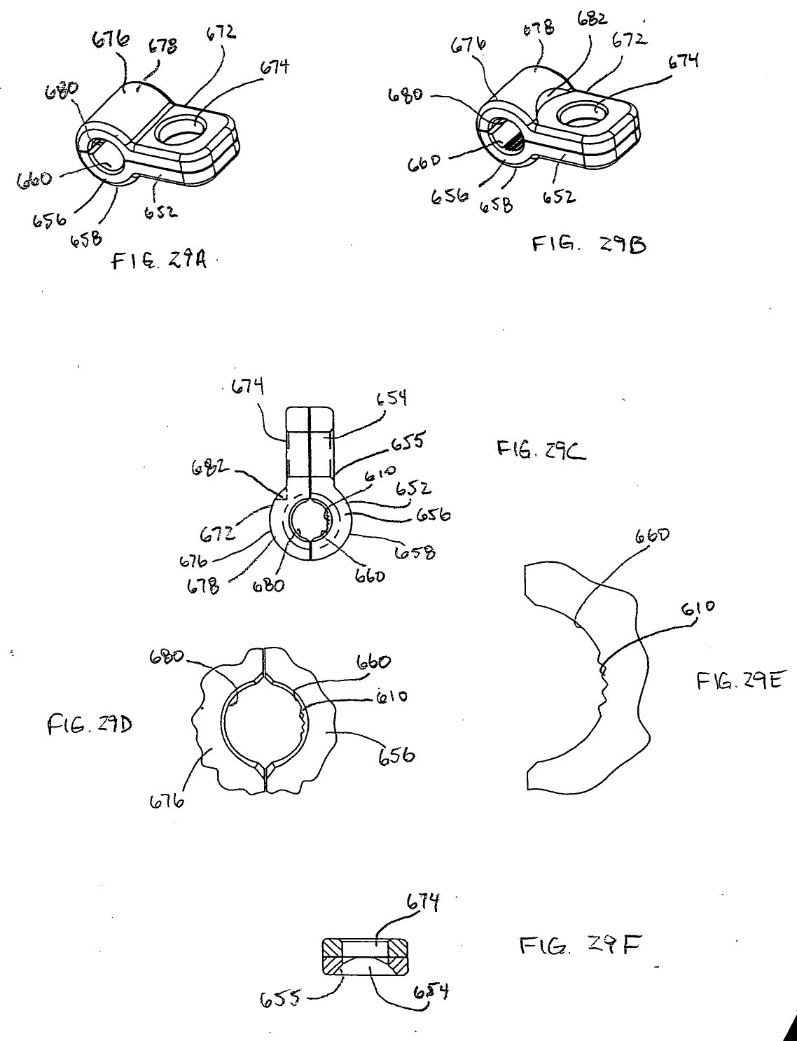

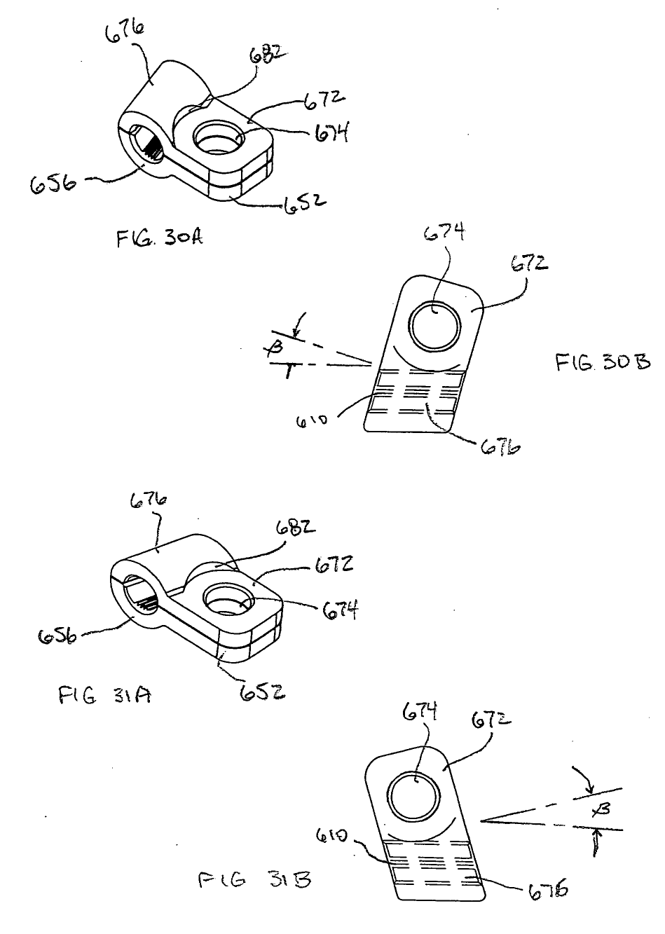

A preferred jam nut 100 is shown in FIGS. 28A-B. The jam nut 100 is a hex jam nut, although twelve-point or other jam nuts are also suitable. The jam nut 100 preferably has rounded edges 102, which may minimize tissue irritation, e.g., of tissue overlying the nuts 100 after implantation of the system. In addition, the jam nuts 100 may include a crimpable rim 104, which may be crimped when the nuts are tightened to a desired torque, e.g., to prevent subsequent loosening of the nuts. The jam nut 100 is also provided with an enlarged flange 106 extending on the bottom portion of the jam nut 100. The enlarged flange 106 extends below the gripping surfaces 108 of the jam nut, forming the bottom surface of the fastener. The enlarged flange 106 forms the surface that meets, for example, a facing surface of the clamp assembly 16 when the spinal fixation system is assembled. The provision of an enlarged flange 106 allows the fastening force applied by the fastener to be applied over a broader surface area relative to a similar fastener that does not include the enlarged flange 106. This provides a more secure mechanism for fastening a clamp assembly to an anchor screw.

The clamp assemblies and rods of the present spinal fixation systems will now be described. In the present embodiments, the clamp assemblies and rods are modified from the previously described embodiments to provide an alternative mechanism for obtaining a firm gripping purchase between the clamp assemblies and the rods. To this end, each of the rods described below is provided with elongated serrations over at least a portion of the peripheral surface of the rod. Similarly, the clamp assemblies are provided with grooves over at least a portion of the interior engagement surfaces that are intended to mate with the serrations formed on the rod to thereby improve the gripping strength of the finished assembly.

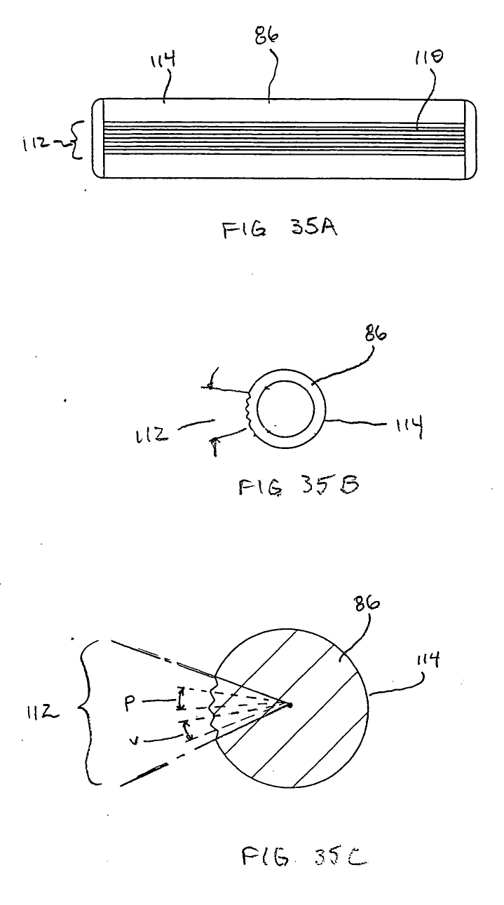

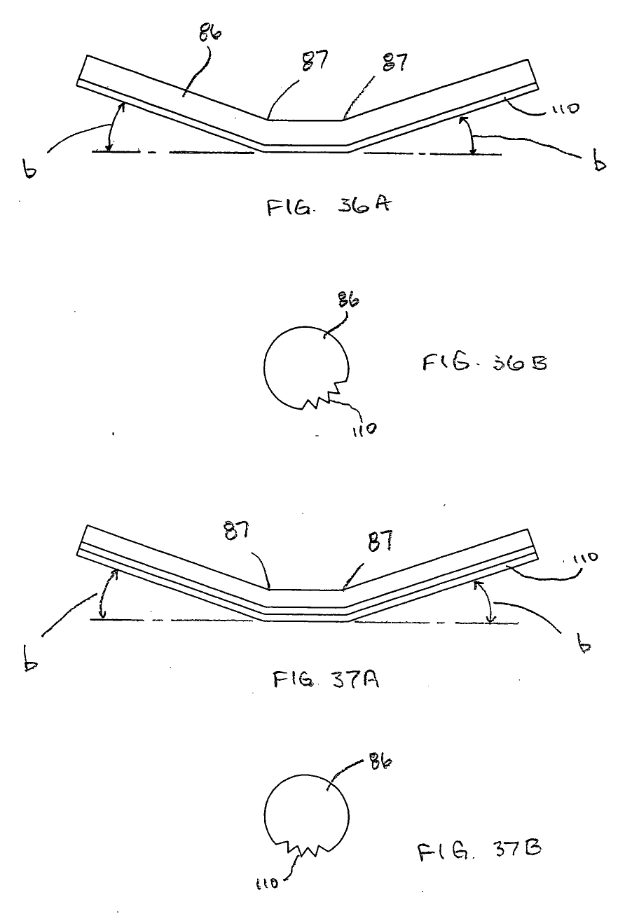

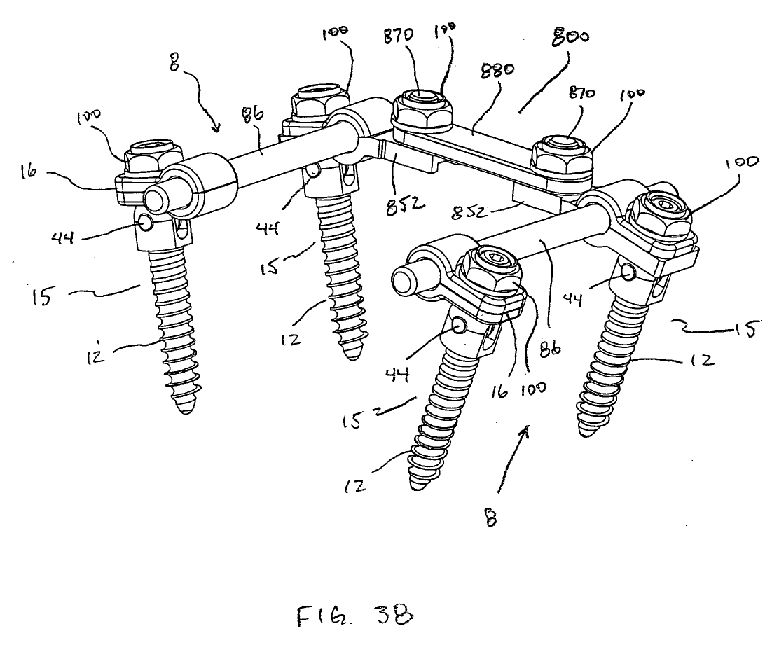

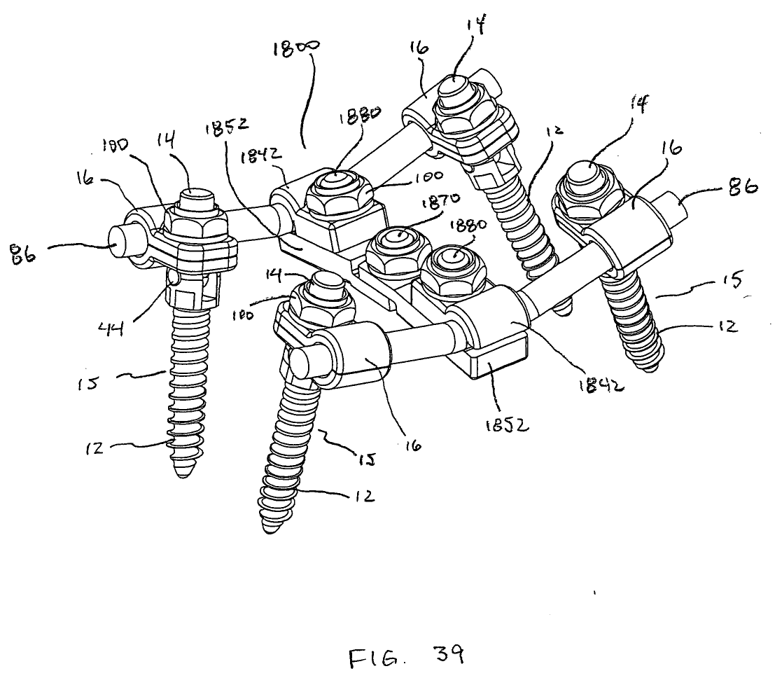

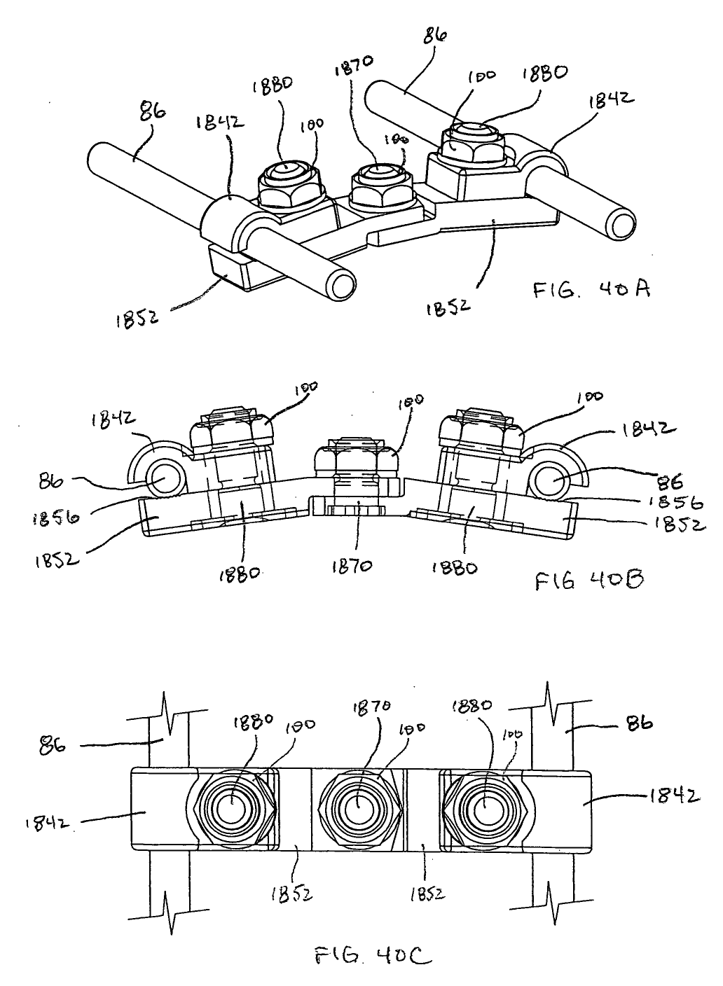

For example, the rod 86 in the spinal fixation system shown in FIG. 23A includes limited number of serrations formed on the lower-facing surface of the rod (not shown in FIG. 23A). The construction of the rod is shown more clearly in FIGS. 35A-C. The rod 86 is a substantially rigid elongate member, e.g., a solid rod, having a generally round cross-section. A plurality of serrations 110 are formed on the outer surface of the rod. The serrations 110 may extend over the entire length of the rod 86. However, in the illustrated embodiment, the serrations 110 extend over only a limited section 112 of the periphery of the rod, whereas the remaining section 114 of the rod has a generally smooth, unserrated finish.

The serrations formed on the serrated section of the rod will be described in more detail in relation to FIG. 35C. In this embodiment, the serrations are uniformly spaced and comprise three peaks and four valleys within the serrated section. More or fewer peaks and valleys may be used. The serrations extend uniformly through the serrated region of the rod, which comprises an angular distance covering less than 25% of the rod, and preferably less than 10% of the rod. In the preferred embodiment shown in FIG. 35C, the serrated region comprises an angular distance of about 48°, which accounts for about 7.5% of the circumference of the rod.

The distance between each peak may be defined by the peak separation angle, “p”, which is the angle formed between imaginary lines drawn from the center of the rod through two adjacent peaks. Preferably, the peak separation angle is within the range of from about 3° to about 24°, and more preferably within the range of from about 6° to about 18°. In the embodiment shown in FIG. 35C, the peak separation angle is approximately 12°. Similarly, the valley separation angle, “v”, is the angle formed between imaginary lines drawn from the center of the rod through two adjacent valleys. Preferably, the valley separation angle is also within the range of from about 3° to about 24°, and more preferably within the range of from about 6° to about 18°. In the embodiment shown in FIG. 35C, the valley separation angle is also approximately 12°.

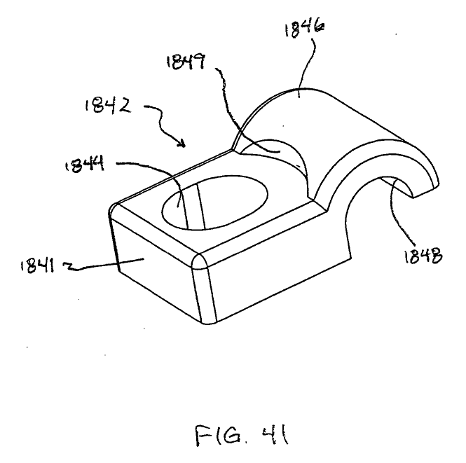

FIGS. 36A-B and 37A-B illustrate partially serrated rods 86 that are provided with two bends 87 in each rod 86. As noted above, bends and/or curves may be formed in a rod 86 in order to conform to the natural curvature or lordosis of the anatomy encountered. The angles, b, formed by each of the bends will depend upon the particular curvature or anatomy encountered, but typically will fall between about 0° to about 45°. As shown by the end views (FIGS. 36B and 37B), the serrations 110 are equal in both rotation directions per side.

An alternative serrated rod is included in the spinal fixation system shown in FIG. 23B. In that embodiment, the rod 86 has longitudinal serrations 110 formed over the entire peripheral surface of the rod. A more detailed illustration of the fully serrated rod 86 is shown in FIGS. 34A-B. As shown there, the serrations 110 extend longitudinally over the length of the rod 86, and are formed over the entire circumferential surface of the rod. Additional descriptions of similar fully serrated rods can be found in U.S. Pat. No. 4,653,481, which is hereby incorporated by reference.

The present inventor has found that the partially serrated rod (FIGS. 35A-C) provides a number of advantages over the fully serrated rod (FIGS. 34A-B). One such advantage is due to the method used to form the serrations on such rods. Typically, the serrations are formed by creating a plurality of races in the surface of an otherwise smooth cylindrical rod. The machining processes used to form the races removes material from the surface of the rod to create grooves in the surface. These processes have a tendency to create stress fractures in the rod material, and to otherwise weaken the structural integrity of the rod. This effect is cumulative, i.e., the tendency to create stress fractures and to weaken the rod increases as additional races are formed in the rod surface. Accordingly, the present preferred partially serrated rod will typically possess greater structural integrity, fewer stress fractures, and will be stronger than the comparable fully serrated rods of the prior art.

In addition, the present inventor has found that the clamping force obtained in the spinal fixation systems utilizing the partially serrated rods is typically greater than that obtained in similar systems utilizing the prior art fully serrated rods.

As described above, the spinal fixation systems shown in FIGS. 23A-B include clamp assemblies 16 for attaching the rods 86 to the anchor screws 15. Additional details relating to the clamp assemblies are shown in FIGS. 29A-E. In several preferred embodiments, the described clamp assemblies are adapted to effectively receive and retain the partially serrated rod. In addition, the described clamp assemblies include features and components that provide a lower profile than the clamp assemblies described previously.

Turning to FIGS. 29A-F, each clamp assembly includes an upper clamp portion 672 and a lower clamp portion 652. The upper clamp portion 672 includes a first passage 674 therethrough and, similarly, the lower clamp portion 652 includes a first passage 654 therethrough. When the upper clamp portion 672 and lower clamp portion 652 are brought together, (as shown, for example, in FIGS. 29A and 29B), the first passages 674, 654 are in alignment to define a passageway having a size and shape to allow passage of the upper end of a swing bolt 14. In the embodiment shown in FIGS. 29A-F, the first passages 674, 654 have a circular cross-section to accommodate a cylindrical swing bolt 14. In alternative embodiments, not shown, the first passages 674, 654 may be formed having a non-circular cross-section to accommodate a non-cylindrical swing bolt, such as those described previously herein.

The upper clamp half 672 includes a saddle portion 676 at its end opposite the first passage 674. Similarly, the lower clamp half 652 includes a saddle portion 656 at its end opposite the first passage 654. The saddle portions 676, 656 are each generally semi-cylindrically shaped members having a convex outer surface 678, 658 and a concave inner surface 680, 660. When the upper clamp portion 672 and lower clamp portion 652 are brought together, (as shown, for example, in FIGS. 29A and 29B), the inner surfaces 680, 660 of the saddle portions face toward one another to define a passageway having a size and shape to receive and retain a rod 86. The inner surfaces 680, 660 thereby form the engagement surfaces of the clamp assembly for engaging the rod 86.

The lower clamp half 652 includes a spherical seat 655, as best seen in FIG. 29F. The spherical seat 655 is the surface that engages the shoulder 28 of the screw 12 of the anchor screw 15, which is also preferably spherical. This structure allows the shoulder 28 to freely pivot in the seat 655 of the lower clamp 652. This feature, when combined with the interaction of the cylindrical upper end of the swing bolt 14 and the cylindrical passageway formed by the first passages 674, 654 of the upper and lower clamps, allows any compound angle to be achieved between the screw 12 and the rod 86 (see FIG. 22) when the spinal fixation system is installed.

The inner surface 660 of the lower clamp portion 652 includes a section having a plurality of serrations 610 formed thereon. The serrations 610 comprise raised peaks formed on the inner surface 660 of the lower clamp portion 652. The serrations 610 preferably have the same or a similar profile as the serrations 110 formed on the rod 86. More particularly, in the preferred embodiment, the serrations 610 will be formed over a portion of the inner surface 660 of the lower clamp portion 652 that is the same size as the portion of the outer surface of the rod 86 that is provided with serrations. As noted above, this comprises less than about 25%, and preferably less than about 10%, of the surface area of the rod 86. Because the lower clamp portion 652 only engages one-half of the rod 86, the comparable surface area of the lower clamp portion inner surface 660 is less than about 50%, and preferably less than about 20%. In addition, in the preferred embodiment, the size, shape, and relative spacing of the peaks and valleys formed by the serrations 610 are preferably the same as, or similar to, the size, shape, and relative spacing of the peaks and valley formed by the serrations 110 on the surface of the rod 86.

Turning again to FIG. 29B, a carve-out 682 is formed on the outer surface 678 of the upper clamp portion 672. For comparison, no carve-out 682 is formed on the lower clamp portion 672 illustrated in FIG. 29A. The carve-out 682 has a semi-cylindrical shape and is intended to accommodate a portion of the flange 106 of the jam nut 100 when the jam nut 100 is installed onto the threaded region 40 of the swing bolt 14. The carve-out 682 thereby allows the jam nut 100 to assume a lower profile than would be otherwise required to accommodate the jam nut 100. As a result, the upper surface of the jam nut 100 preferably does not extend any higher than the upper edge of the upper clamp portion 672.

FIGS. 33A-F show a clamp assembly that is similar to that described above in relation to FIGS. 29A-F. The clamp assembly shown in FIGS. 33A-F, however, includes serrations 610 formed on both the lower clamp inner surface 660 and the upper clamp inner surface 680. This construction is designed, for example, to accommodate use of a rod 86 having serrations 110 on opposed sides of the rod 86.

FIGS. 30A-B and 31A-B show additional alternative embodiments of the lower and upper clamp portions wherein the rod 86 recesses are at a different angle than those of FIGS. 29A-F or 33A-F and define an angle β similar to the embodiments shown in FIGS. 20-21 and in FIG. 9d. The clamp assemblies are otherwise substantially identical to those described above.

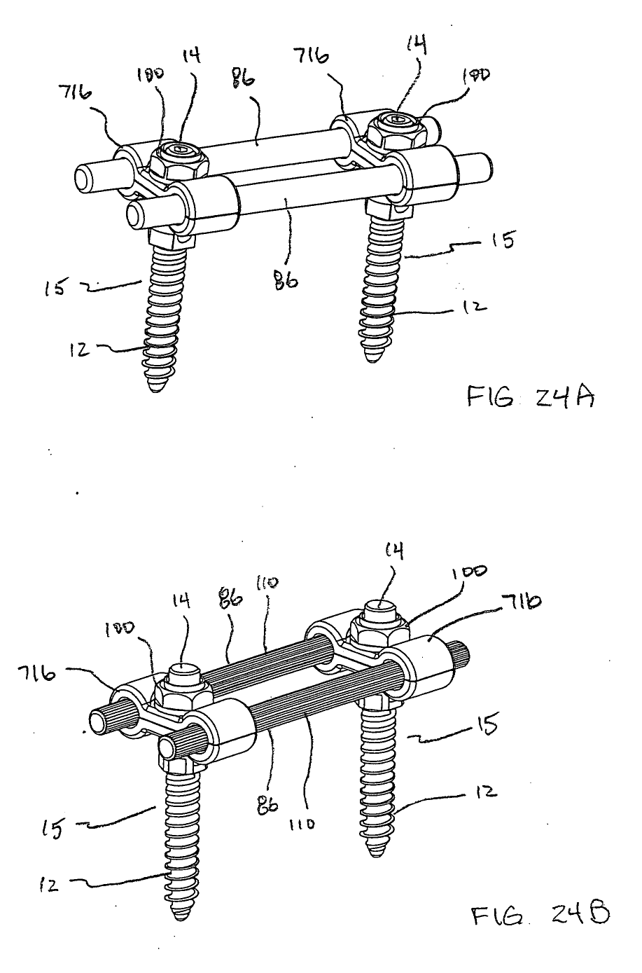

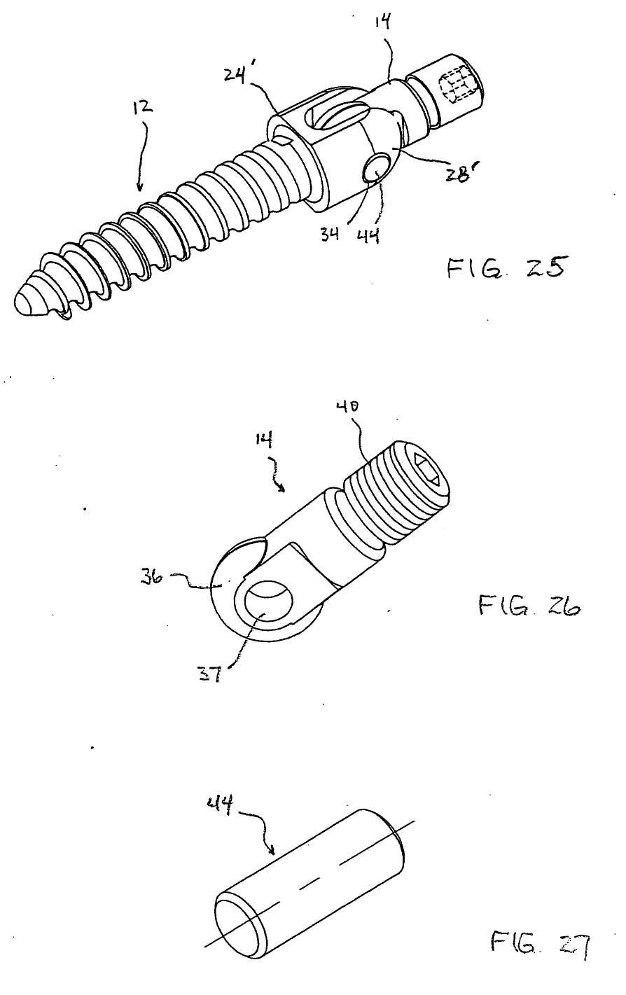

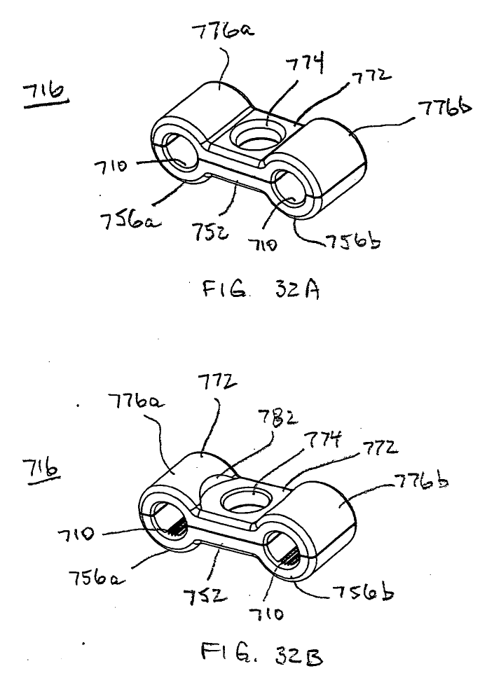

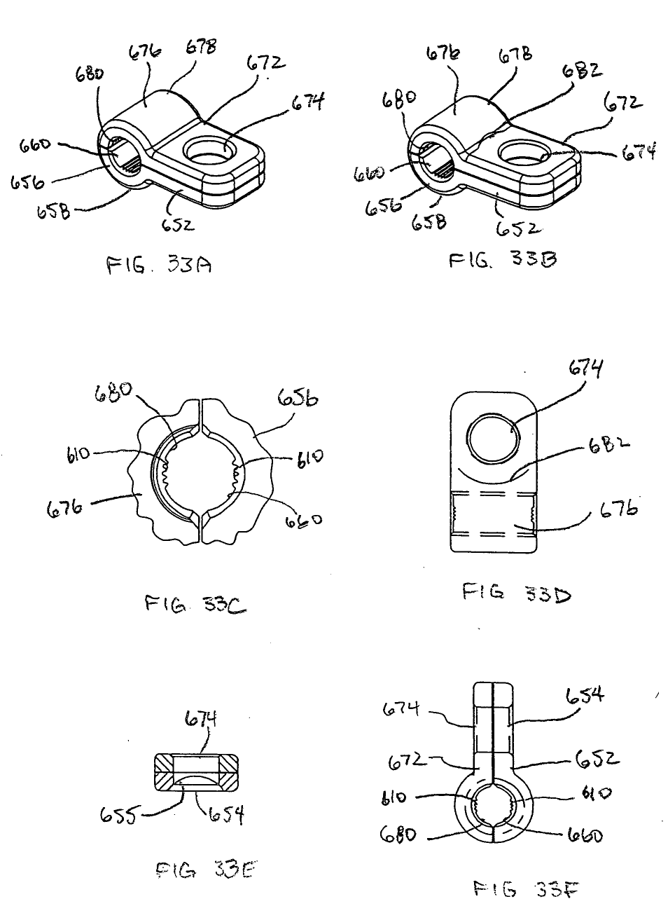

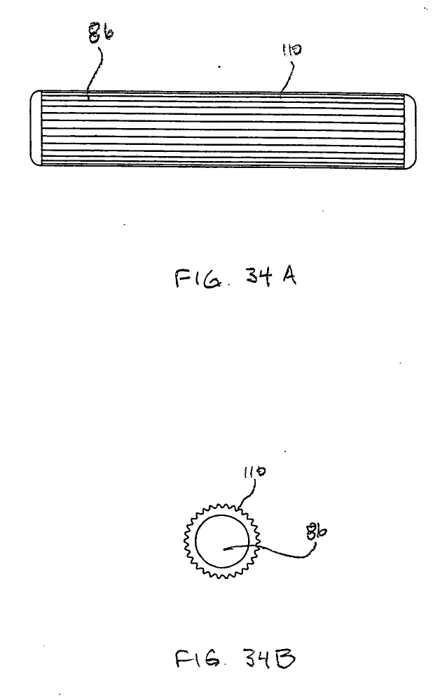

FIGS. 24A-B show alternative embodiments of the spinal fixation system that include provision of a double rod assembly. The double-rod assembly is facilitated by provision of a double-rod clamp assembly 716, as best seen in FIGS. 32A-B. The double-rod clamp assembly 716 includes an upper clamp portion 772 and lower clamp portion 752, each having a first passage 774, 754, which together define a passageway for receiving the upper end of the swing bolt 14. The upper clamp 772 and lower clamp 752 also each include a pair of saddle portions 776a-b, 756a-b on opposed sides of the first passages, the saddle portions together defining a second passageway for receiving and retaining the two rods 86. The inner surfaces of the saddle portions 776a-b, 756a-b are provided with serrations 710 adapted to mate with the serrations 110 contained on the respective rods 86. In addition, the double-rod clamp assembly 716 shown in FIG. 32B includes a carve-out 782 to accommodate the jam nut 100 and to provide a relatively lower profile for the fixation system.

The double-rod spinal fixation systems shown in FIGS. 24A-B provide an additional measure of structural rigidity and strength to the system, while occupying an additional amount of space due to the provision of the second rod. Accordingly, the double-rod systems are preferably used in those situations in which the having a relatively stronger, more rigid system is preferable, and in which the additional space is available.

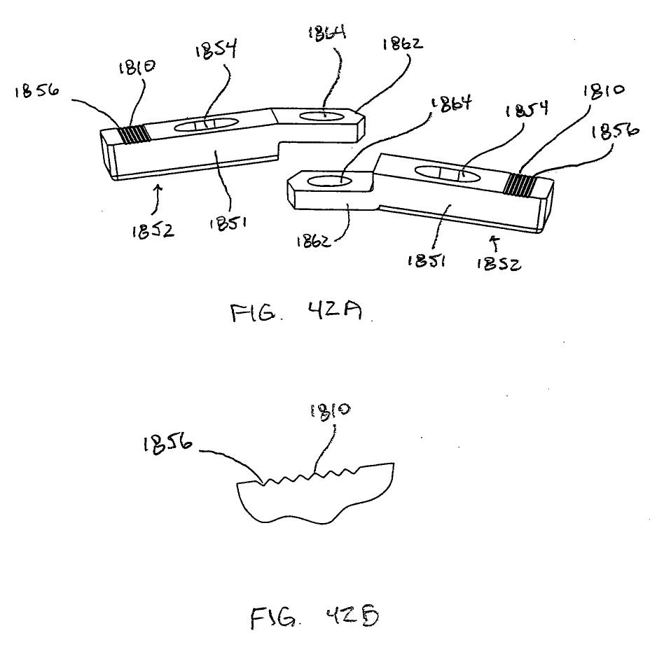

Turning now to FIGS. 38 and 39, the spinal fixation systems described herein may be supplemented with one or more cross-linking structures. FIG. 38 shows an end cross-linking structure 800 attached to the ends of a pair of spinal fixation systems, and FIG. 39 shows a mid cross-linking structure 1800 attached to a mid-section of another pair of spinal fixation systems. These cross-linking structures provide additional structural strength to their associated spinal fixation systems and provide additional functionality to the systems to the extent that additional distraction or attraction forces may be applied through utilization of such cross-linking structures. The structures and component parts of the preferred cross-linking structures will now be described.

Turning first to the end cross-linking structure 800 shown in FIG. 38, the system includes two spinal fixation systems such as those described above in relation to FIGS. 22-37, with a modification to the clamping assemblies associated with the anchor screws at a first end of each of the fixation systems. At the second ends, each spinal fixation system includes an anchor screw 12, a swing bolt 14 attached to the anchor screw by a pin 44, and a clamp assembly 16 attached to the swing bolt 14 by a fastener, such as a jam nut 100. Each of the clamp assemblies 16 is attached to a rod 86, which is preferably partially serrated. The clamp assemblies 16 are preferably provided with partially serrated engagement surfaces adapted to mate with the partially serrated rod 86, as described above.

Turning again to the first ends of the spinal fixation systems, the system components are generally the same as those described above, except that the lower clamp of each of the clamp assemblies is replaced with a lower clamp cross link 852. (See also FIGS. 45A-B, 46A-B). The lower clamp cross link 852 includes a first passage 854 adapted to receive the swing bolt, and a saddle portion 856 forming a partially serrated inner surface 860 adapted to engage a mating serrated surface of the rod 86. A cross-brace attachment platform 862 extends laterally away from the saddle portion 856 on the side opposite the first passage 854. The attachment platform 862 includes a second passageway 864 adapted to receive and retain a threaded clamp pin 870 therethrough. A cross bracing link 880 is placed over the clamp pin 870 extending from each of the attachment platforms 862, and a fastener, such as a jam nut 100, is threaded onto the clamp pin 870 to fixedly attach the cross bracing link 880 to each of the attachment platforms 862 of the lower clamp cross links 852.

As noted above, additional details of the lower clamp cross link 862 are shown in FIGS. 45A-B and 46A-B. The lower clamp cross link 862 includes a spherical seat 855 formed at the lower end of the first passage 854. The spherical seat 855 is similar to those described above in relation to the lower clamp portions 52 shown in, for example, FIGS. 18A and 18D. The embodiment shown in FIGS. 45A-B includes four serrations 810 formed on the inner surface 860 of the saddle portion 856, although more or fewer serrations are also possible. For example, a particularly preferred embodiment includes only two serrations 810, such as shown in FIGS. 46A-B. The size, shape, and spacing of the serrations 810 are selected in order to optimize the gripping strength of the clamp assembly to the rod 86. In the preferred embodiment, the two serrations are shaped identically to those contained on the associated rod 86, and are have a peak angle of 12° and a valley angle of 12°.

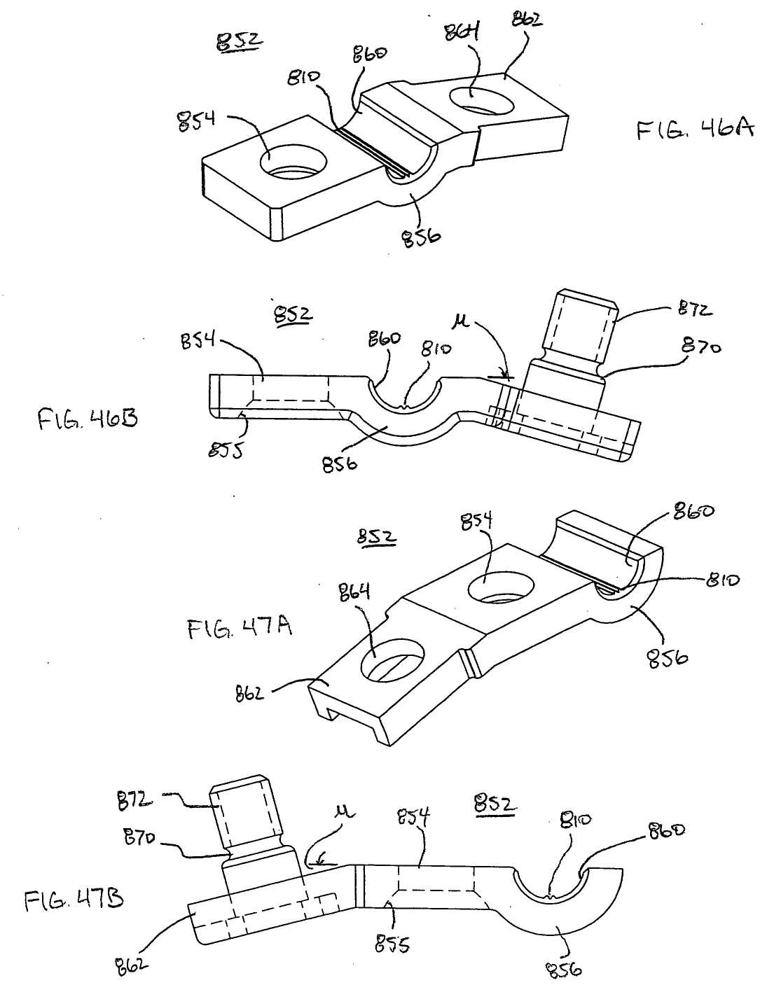

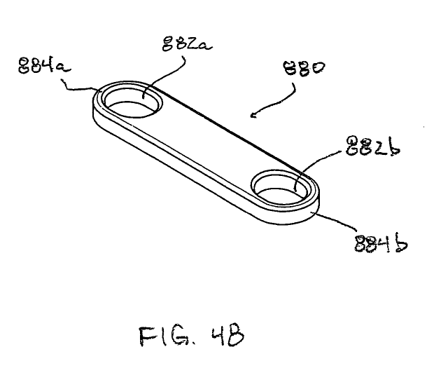

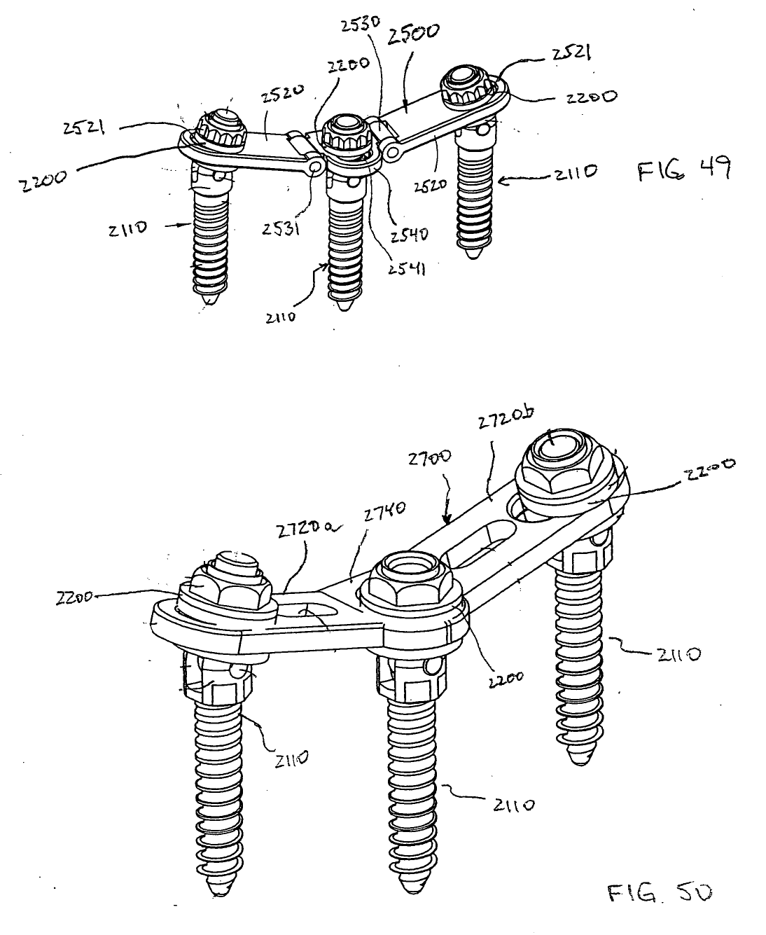

An alternative embodiment of the lower clamp cross link 852 is shown in FIGS. 44A-B. In the alternative embodiment, the first passage 854 is located between the saddle portion 856 and the cross-brace attachment platform 862. Other than this substitution, the lower clamp cross link 852 is structurally identical to the embodiment described above in relation to FIGS. 45A-B. The purpose for the re-orientation of the first passage 854 and the saddle portion 856 is to accommodate a crosslinking system for a pair of spinal fixation systems in which the anchor screw 15 is located on the same side of the rod 86 as is the cross bracing link 880, i.e., in the area located between the two rods 86 of the pair of spinal fixation systems. This orientation is not shown in the drawings, but will be readily apparent to the person of ordinary skill in the art from the structure of the lower clamp cross link 852 described above in relation to FIGS. 44A-B.

Additional alternative embodiments of the lower clamp cross link 852 are shown in FIGS. 46A-B and 47A-B. The lower clamp cross link 852 embodiment shown in FIGS. 46A-B is identical to that described above in relation to FIGS. 45A-B, but it is provided with a pre-formed bend between the saddle portion 856 and the cross-brace attachment platform 862. The bend, having an angle μ, is pre-formed in the lower clamp cross link 852 in order to conform to the natural curvature or lordosis of the anatomy encountered. Although the bend angle μ is selected based upon the clinical indication, the embodiment shown in FIGS. 46A-B has a bend angle of approximately 15°. The cross-linking structure shown in FIG. 38 illustrates a pair of lower clamp cross links 852 having pre-formed bends. Similarly, the lower clamp cross link 852 shown in FIGS. 47A-B is identical to that described above in relation to FIGS. 44A-B, but is provided with a pre-formed bend between the central portion and the cross-brace attachment platform 862. Again, although the bend angle μ is selected based upon the clinical indication, the embodiment shown in FIGS. 47A-B has a bend angle of approximately 15°.

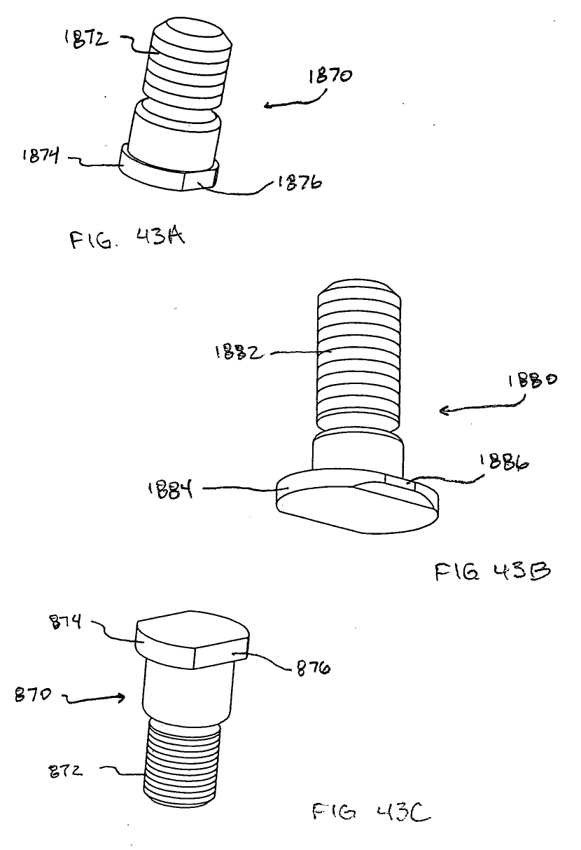

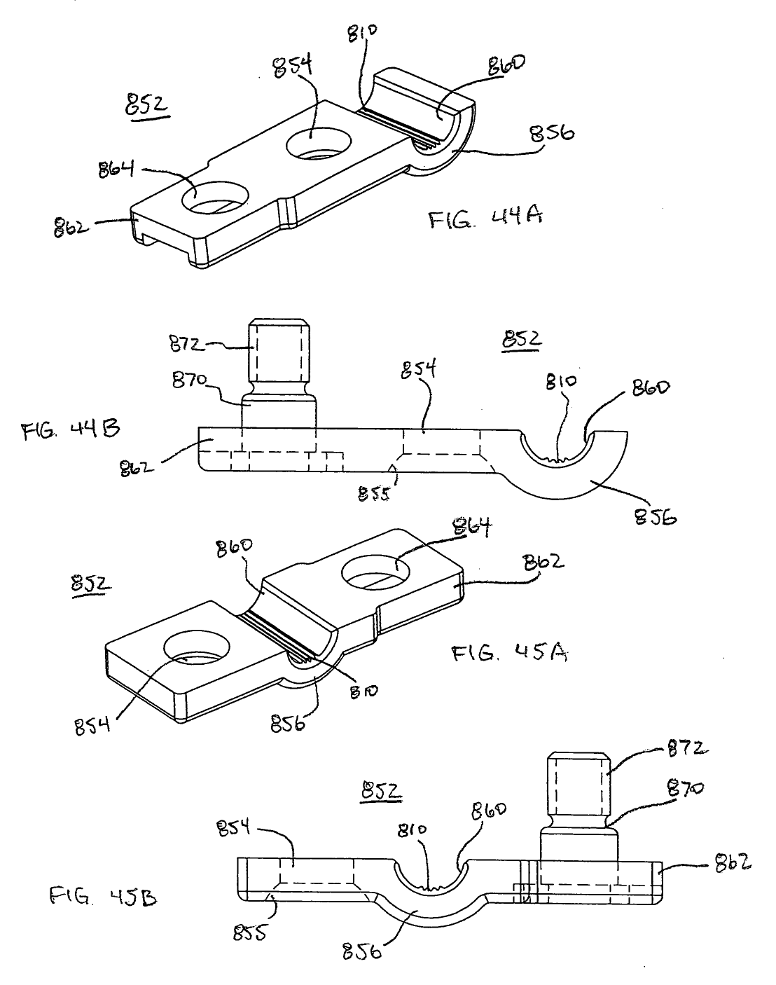

The clamp pin 870 is shown in more detail in FIG. 43C. The clamp pin 870 includes a threaded region 872 at a first end and a flange 874 at the opposite end. The threads of the threaded region 872 are of a size and pitch adapted to engage the jam nut 100 shown attached to each of the clamp pins in FIG. 38. The flange 874 is partially cylindrical, having a pair of opposed flat regions 876 formed thereon. The size and shape of the flange 874 are such that the flange fits within a slot formed on the underside of the cross-brace attachment platform 862 of each of the lower clamp cross links 852. In this way, the flat regions 876 of the flange 874 engage the sides of the slot to prevent rotation of the clamp pin 870 when the jam nut 100 is attached to the clamp pin 870.

The cross bracing link 880 is shown in more detail in FIG. 48. The cross bracing link 880 is a flat, elongated member having a bolt passage 882a-b formed near each end thereof. The ends 884a-b of the cross-bracing link 880 are generally rounded to reduce or prevent tissue and nerve irritation. The length of the cross bracing link 880 is selected based upon the anatomy encountered in order to extend between and interconnect the lower clamp cross links 852 formed on a pair of cross-linked spinal fixation systems.

Turning now to the mid cross-linking structure 1800 shown in FIGS. 39 and 40A-C, the system includes two spinal fixation systems such as those described above in relation to FIGS. 22-37, each of which spinal fixation systems includes a rod 86 having a pre-formed bend at approximately its mid-length. A mid cross-link structure 1800 is attached to each of the rods 86 of the two spinal fixation systems near the mid-point of each of the rods 86, but not directly upon the bends formed in the rods. The mid cross-link structure 1800 includes an elongated cross link 1852 and an upper clamp 1842 attached to each of the two rods 86, with the elongated portions of the cross links 1852 directed toward each other and toward the space between the two spinal fixation systems. A sliding clamp stud 1880 extends through the bolt passages formed by each of the upper clamps 1842 and the cross links 1852, and a jam nut 100 is threaded onto the end of each of the sliding clamp studs 1880, thereby attaching the mid cross-link structure to each of the rods 86 of the two spinal fixation systems. A center clamp stud 1870 extends through the bolt passage formed by the joining of the attachment platforms 1862 of each of the cross links 1852, and a jam nut 100 is threaded onto the end of the center clamp stud 1870, thereby attaching together the two cross links 1852 of the mid cross-link structure 1800.

The upper clamp 1842 is shown in more detail in FIG. 41. The upper clamp 1842 includes a base portion 1841 having a first passage 1844 formed therethrough. The first passage 1844 is preferably constructed to have an oval cross-section, rather than circular, for purposes to be described below. The upper clamp 1842 further includes a saddle portion 1846, the inner surface 1848 of which is adapted to engage a rod 86 of a spinal fixation system. As shown in FIG. 41, the upper clamp 1842 also preferably includes a semi-cylindrical carve-out 1849 on its upper surface over a portion of the saddle portion 1846. The carve-out 1849 is of a size and shape to accommodate a flange 106 of a jam nut 100 to facilitate a low profile construction of the mid cross-linking structure 1800.

A pair of cross links 1852 are shown in more detail in FIGS. 42A-B. Each cross link 1852 includes an elongated main body 1851 having a first passage 1854 formed therethrough. The first passage 1854 is preferably constructed to have an oval cross-section, rather than circular, for purposes to be described below. Each cross link 1852 further includes an engagement surface 1856 formed on the upper surface of the main body 1851 adjacent to the first passage 1854. The engagement surface 1856 includes a plurality of serrations 1810 formed thereon, which serrations 1810 are adapted to engage similar serrations 110 formed on the external surface of the rod 86 of the associated spinal fixation system. The size, shape, and spacing of the serrations 1810 are shown in the close-up view in FIG. 42B. The serrations 1810 are preferably formed over a sufficiently wide area of the upper surface of the main body 1851 of the cross link 1852 to facilitate the available adjustability of the cross link, as described below. The cross links 1852 also each include an attachment platform 1862 extending from the end of the main body 1851 portion opposite the engagement surface 1856. Each attachment platform 1862 includes a second bolt passageway 1864 therethrough. The attachment platform 1862 is preferably about one-half the thickness of the main body portion 1851 and may extend from the upper half of the main body portion 1851 (see left side of FIG. 42A) or the bottom half of the main body portion 1851 (see right side of FIG. 42A). Each pair of cross links 1852 includes one of each the two examples shown in FIG. 42A.

The center clamp stud 1870 is shown in more detail in FIG. 43A. The center clamp stud 1870 includes a threaded region 1872 at a first end and a flange 1874 at the opposite end. The threads of the threaded region 1872 are of a size and pitch adapted to engage the jam nut 100 shown attached to the center clamp stud 1870 in FIGS. 39 and 40A-C. The flange 1874 is partially cylindrical, having a pair of opposed flat regions 1876 formed thereon. The size and shape of the flange 1874 are such that the flange fits within a slot formed on the underside of the attachment platform 1862 of the lower cross link 1852. In this way, the flat regions 1876 of the flange 1874 engage the sides of the slot to prevent rotation of the center clamp stud 1870 when the jam nut 100 is attached to the center clamp stud 1870.

The sliding clamp stud 1880 is shown in more detail in FIG. 43B. The sliding clamp stud 1880 includes a threaded region 1882 at a first end and a flange 1884 at the opposite end. The threads of the threaded region 1882 are of a size and pitch adapted to engage the jam nut 100 shown attached to the each of the sliding clamp studs 1880 in FIGS. 39 and 40A-C. The flange 1884 is partially oval-shaped, having a pair of opposed flat regions 1886 formed thereon. The size and shape of the flange 1884 are such that the flange fits slidably within a slot formed on the underside of the cross link 1852, which slot is in communication with the first passage 1854 of the cross link. In this way, the flat regions 1876 of the flange 1874 engage the sides of the slot to prevent rotation of the sliding clamp stud 1880 when the jam nut 100 is attached to the sliding clamp stud 1880.

However, the sliding clamp stud 1880 is able to slide within the slot to the extent allowed by the oval shape of the bolt passage formed by the first passages 1854, 1844 of the cross link 1852 and the upper clamp 1842. This degree of freedom of lateral movement allows a degree of adjustability to the system. As best seen in FIG. 40B, the upper clamp 1842 may be adjusted laterally on the upper surface of the cross link 1852 due to the oval shapes of the first passages 1844, 1854. Accordingly, if the rod 86 happens to lie a short distance laterally from the set position, the upper clamp 1842 may be shifted slightly to accommodate the new position. This adjustability may be an important feature when, for example, the mid cross-linking assembly is being installed on a pre-installed pair of spinal fixation systems.

As noted earlier herein, in accordance with another aspect of the present invention, a kit is provided for stabilizing vertebrae relative to one another. Generally, the kit includes a collection of several of the component parts of the spinal fixation systems described herein. For example, a representiative kit includes one or more substantially rigid rods (including rods having no serrations, rods having serrations over at least a portion of the peripheral surface of the rod, and/or fully serrated rods), and a plurality of “C” shaped spacers having a plurality of lengths, the spacers including opposing edges defining a pocket therebetween for receiving the one or more rods therein. The kit may also include a plurality of anchor screw assemblies, the anchor screw assemblies including anchor screws and a plurality of clamp assemblies for receiving the one or more rods therein. The anchor screws and clamp assemblies may comprise any one or combination of the embodiments of these components described herein.

Optionally, the kit may also include a tool for crimping at least a portion of the opposing edges of the spacers around the one or more rods to secure the spacers to the rods. In another option, the kit may include an apparatus for bending the one or more rods, e.g., to conform substantially to a natural curvature of a patient's spinal column being treated.

In yet another option, the kit may include one or more flat, elongated, “plate” shaped connecting beams, such as those described in co-pending application Ser. No. 11/733,708, (“the '708 application”), which has been incorporated by reference into the present application. Representative examples of such connecting beams and of spinal fixation systems incorporating such connecting beams are illustrated in FIGS. 49 through 51, which are reproduced from drawings contained in the '708 application.

FIG. 49, for example, illustrates a spinal fixation system that includes a multi-level hinged connecting beam 2500. The multi-level connecting beam includes three primary components, a pair of side-portions 2520 and a center-portion 2540. Each of the side-portions 2520 includes an elongated slot 2521 formed near its end opposite the center-portion for the purpose of engaging the clamp assembly 2200 and the anchor screw assemblies 2110. The center-portion 2540 also includes an elongated slot 2541, which is preferably offset by about 900 from the direction of elongation of the slots 2521 on the side-portions 2520. The center-portion elongated slot 2541 is intended to engage the clamp assembly 2200 and the third anchor assembly 2110. Each of the two side-portions 2520 of the connecting beam is attached to an opposed side of the center-portion by a hinge 2530 and hinge pin 2531.

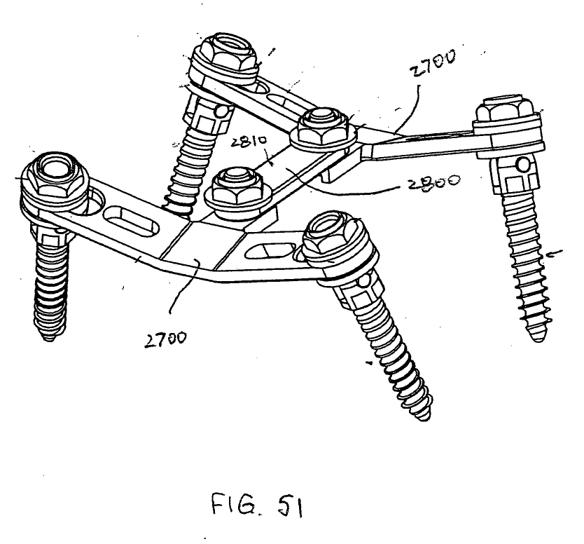

Turning to FIG. 50, another spinal fixation system embodiment includes a pre-formed connecting beam 2700. The pre-formed connecting beam 2700 may be used with two or more anchor screw assemblies 2110. The pre-formed connecting beam 2700 includes a plurality of integrated segments, with each segment extending in a plane that forms a connecting angle with the plane of the adjacent segment. The connecting angle is preferably an acute angle in the range of from about 0° to about 25°, depending upon the clinical need. For example, the pre-formed connecting beam shown in FIG. 25 includes three segments: a center segment 2740 and a pair of end segments 2720a, 2720b. Each of the end segments includes an elongated slot formed near its end opposite the center segment for the purpose of engaging the clamp assembly 2200 and the anchor screw assemblies 2110. The center segment also includes an elongated slot, which is preferably offset by about 90° from the direction of elongation of the slots on the end segments. The center segment elongated slot is intended to engage the clamp assembly 2200 and the third anchor assembly 2110. The pre-formed connecting beam may be adapted to interconnect two, three, or more anchor screw assemblies.

Turning next to FIG. 51, the spinal fixation systems described herein may also include one or more cross-brace members 2800 extending between and interconnecting two or more connecting beams. The preferred cross-brace member comprises a flat, elongated member 2810, each end of which is attached to a separate connecting beam. The flat, elongated member 2810 is preferably formed of the same materials used to make the connecting beams 2700, described elsewhere herein. Additionally, the flat, elongated member 2810 preferably has cross-sectional dimensions (e.g., width, plate thickness) the same as or similar to the connecting beams. However, because it performs different functions and bears different types of loads, the flat, elongated member 2810 may also have very different dimensions than those of the connecting beams.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the spirit and scope of the appended claims.

What is claimed is:

1. A spinal fixation system comprising
a first anchor screw assembly having a first screw having a threaded portion configured to be screwed into a first vertebrae, the first anchor screw assembly including a first cylindrical swing bolt pivotably coupled to the first screw, and the first anchor screw assembly comprising a first clamp assembly having a cylindrical opening disposed on the first swing bolt of the first anchor screw assembly, the first clamp assembly having a first passage therethrough,
a second anchor screw assembly comprising a second screw having a threaded portion configured to be screwed into a second vertebrae adjacent the first vertebrae, the second anchor screw assembly including a second cylindrical swing bolt pivotably coupled to the second screw, and the second anchor screw assembly comprising a second clamp assembly having a cylindrical opening disposed on the second swing bolt of the second anchor screw assembly, the second clamp assembly having a second passage therethrough, and
a first elongate member receivable in the first and second passages, the first elongate member having a plurality of longitudinal serrations extending over less than about 25% of the cross-sectional periphery thereof.

2. The spinal fixation system of claim 1 wherein said longitudinal serrations on said first elongate member extend over less than about 10% of the cross-sectional periphery thereof.

3. The spinal fixation system of claim 2, wherein said serrations on said first elongate member comprise no more than four peaks and four valleys.

4. The spinal fixation system of claim 1 wherein the first and second clamp assemblies each comprise upper and lower clamp portions defining the respective passages therebetween.

5. The spinal fixation system of claim 4, wherein at least one of said upper and said lower clamp portions comprises an engagement surface having a plurality of serrations formed on a portion thereof.

6. The spinal fixation system of claim 5, wherein said serrations on said at least one of said upper and said lower clamp portions comprise no more than four peaks and four valleys.

7. The spinal fixation system of claim 1 wherein each of the first and second screws comprises a head portion opposite the threaded portion comprising a spherical shoulder, the spherical shoulder being adapted to mate, pivot and rotate with respect to the respective first and second clamp assemblies.

8. The spinal fixation system of claim 1 wherein each swing bolt comprises a threaded region opposite the respective first and second screws, and wherein a fastener is threadable onto the threaded region for securing the respective clamp assemblies on the swing bolts.

9. The spinal fixation system of claim 1 wherein the first elongate member is at least partially curved, and the passageways in the first and second clamp assemblies are configured for receiving the first elongate member.

10. The spinal fixation system of claim 1, further comprising
a third anchor screw assembly having a third screw having a threaded portion configured to be screwed into a third vertebrae, the third anchor screw assembly including a third cylindrical swing bolt pivotably coupled to the third screw, and the third anchor screw assembly comprising a third clamp assembly having a cylindrical opening disposed on the third swing bolt of the third anchor screw assembly, the third clamp assembly having a third passage therethrough,
a fourth anchor screw assembly comprising a fourth screw having a threaded portion configured to be screwed into a fourth vertebrae adjacent the third vertebrae, the fourth anchor screw assembly including a fourth cylindrical swing bolt pivotably coupled to the fourth screw, and the fourth anchor screw assembly comprising a fourth clamp assembly having a cylindrical opening disposed on the fourth swing bolt of the fourth anchor screw assembly, the fourth clamp assembly having a fourth passage therethrough, and
a second elongate member receivable in the third and fourth passages, the second elongate member having a plurality of longitudinal serrations extending over less than about 25% of the cross-sectional periphery thereof,
wherein said third vertebrae may be the same as either of said first vertebrae or said second vertebrae, or may be a different vertebrae from either of said first vertebrae and said second vertebrae, and wherein said fourth vertebrae may be the same as either of said first vertebrae or said second vertebrae, or may be a different vertebrae from either of said first vertebrae and said second vertebrae, but said third vertebrae is not said fourth vertebrae.

11. The spinal fixation system of claim 10, further comprising a cross-linking structure interconnecting said first anchor screw assembly and said third anchor screw assembly.

12. The spinal fixation system of claim 11, wherein said cross-linking structure comprises:
a first cross link member having a first attachment platform, said first cross link member being attached to said first anchor screw assembly,
a second cross link member having a second attachment platform, said second cross link member being attached to said third anchor screw assembly, and
a cross-brace attached to said first cross link member and attached to said second cross link member.

13. The spinal fixation system of claim 10, further comprising a cross-linking structure interconnecting said first elongate member and said second elongate member.

14. The spinal fixation system of claim 13, wherein said cross-linking structure comprises:
a first cross link member having a first attachment platform, said first cross link member being attached to said first elongate member,
a second cross link member having a second attachment platform, said second cross link member being attached to said second elongate member,
wherein said first attachment platform is attached to said second attachment platform.

15. A kit for stabilizing vertebrae relative to one another, comprising
one ore more substantially rigid rods, each of said rods having a longitudinal axis and having a plurality of longitudinal serrations extending over less than 25% of the cross-sectional periphery thereof,
a plurality of anchor screw assemblies, the anchor screw assemblies comprising anchor screws and a plurality of clamp assemblies for receiving the one or more rods therein, the anchor screw assemblies each including a screw configured to be screwed into a vertebrae and including a cylindrical swing bolt pivotably coupled to the first screw.

16. The kit of claim 15, wherein said longitudinal serrations on each of said one or more rods extend over less than about 10% of the cross-sectional periphery thereof.

17. The kit of claim 16, wherein said serrations on each of said one or more rods comprise no more than four peaks and four valleys.

18. The kit of claim 15, wherein each of the clamp assemblies comprises upper and lower clamp portions defining a passage therebetween.

19. The kit of claim 18, wherein at least one of each of said pair of upper and said lower clamp portions comprises an engagement surface having a plurality of serrations formed on a portion thereof.

20. The kit of claim 19, wherein said serrations on said at least one of each of said pair of upper and said lower clamp portions comprise no more than four peaks and four valleys.

21. The kit of claim 15 wherein each swing bolt comprises a threaded region opposite the respective first and second screws, and wherein a fastener is threadable onto the threaded region for securing the respective clamp assemblies on the swing bolts.

22. The kit of claim 15 wherein each screw comprises a head portion opposite a threaded portion and wherein the head portion comprises a spherical shoulder, the spherical shoulder being adapted to mate, pivot and rotate with respect to a respective clamp assembly.

23. The kit of claim 15, further comprising:
one or more cross-linking structures, the cross-linking structures comprising a plurality of cross-linking members configured to attach to an elongated rod or an anchor screw assembly, and one or more connecting members configured to attach two or more cross-linking members together.

24. The kit of claim 15, further comprising one or more connecting beams.
